(19) United States
(12) Patent Application Publication
(10) Pub. No.: US 2025/0349437 A1
(43) Pub. Date: Nov. 13, 2025

(54) CLINICO-ENDOTHELIAL BIOMARKER RISK MODEL FOR PREDICTING PERSISTENT PEDIATRIC SEPSIS-ASSOCIATED ACUTE RESPIRATORY DYSFUNCTION

(71) Applicant: Children's Hospital Medical Center, Cincinnati, OH (US)

(72) Inventors: James Garrett WILLIAMS, Little Rock, AR (US); Mihir ATREYA, Cincinnati, OH (US)

(73) Assignee: Children's Hospital Medical Center, Cincinnati, OH (US)

(21) Appl. No.: 19/202,787

(22) Filed: May 8, 2025

Related U.S. Application Data
(60) Provisional application No. 63/644,248, filed on May 8, 2024.

(51) Int. Cl. G16H 50/30; G16H 50/20

(52) U.S. Cl. CPC G16H 50/30; G16H 50/20

(57) ABSTRACT

Methods and compositions disclosed herein generally relate to methods of identifying, validating, and measuring clinically relevant, quantifiable biomarkers of diagnostic and therapeutic responses for blood, vascular, cardiac, and respiratory tract dysfunction, particularly as those responses relate to septic shock in pediatric patients. Certain aspects of the invention relate to identifying one or more biomarkers associated with septic shock in pediatric patients in combination with one or more endothelial-derived biomarkers, receiving a dataset comprising biomarker concentrations, wherein the dataset is from a sample obtained from a pediatric patient having at least one indication of septic shock, then determining whether the biomarker concentrations of each of the at least one biomarkers are greater than one or more pre-determined cut-off biomarker concentration, wherein the level of said biomarker correlates with a predicted outcome.

### PRIORITY

The present application claims the benefit of priority under 35 U.S.C. § 119 (e) to U.S. Provisional Application No. 63/644,248, DERIVATION AND VALIDATION OF A CLINICO-ENDOTHELIAL BIOMARKER RISK MODEL TO PREDICT PERSISTENT PEDIATRIC SEPSIS-ASSOCIATED ACUTE RESPIRATORY DYSFUNCTION, filed on May 8, 2024, which is currently co-pending herewith and which is incorporated by reference in its entirety.

### STATEMENT REGARDING FEDERALLY SPONSORED RESEARCH

This invention was made with government support under R 35GM 155165 and R 35GM 126943 awarded by the National Institutes of Health. The government has certain rights to the invention.

### FIELD

Aspects of the present disclosure generally relate to the identification and validation of clinically relevant, quantifiable biomarkers associated with sepsis and septic shock, and in more particular aspects to integration of clinical and endothelial biomarkers for risk prediction in pediatric patients with sepsis-associated acute respiratory dysfunction.

### BACKGROUND

Persistent pediatric sepsis-associated acute respiratory distress syndrome (ARDS) is a common and consequential organ dysfunction associated with high morbidity and mortality in children. However, biological heterogeneity among patients makes identifying those at risk of persistent acute respiratory dysfunction challenging and has impeded the identification of efficacious therapies, with current standard of care being limited to antibiotics and judicious organ support. There is a need for risk-stratification approaches which can enrich pediatric patients in future clinical trials and allow for the effective treatment of such patients using an appropriate therapeutic approach.

### SUMMARY

Various embodiments of the disclosure relate to methods of classifying a patient with septic shock as high risk of sepsis-associated acute respiratory dysfunction (SA ARD) on day 3 (D3) or other than high risk of SA ARD on D3, the methods including: analyzing a dataset including biomarker expression levels of one or more endothelial biomarkers including soluble thrombomodulin (sTM) and vascular cell adhesion molecule 1 (VCAM-1), wherein the dataset is associated with a sample obtained from a pediatric patient with septic shock at a first time point, and wherein the dataset further includes presence or absence of SA ARD on day 1 (D1) and PaO₂ to FIO₂ ratio for the patient; determining whether the biomarker expression levels of each of the one or more endothelial biomarkers are greater than one or more pre-determined cut-off biomarker expression level, and determining whether the PaO₂ to FIO₂ ratio is less than a pre-determined cut-off PaO₂ to FIO₂ ratio; and classifying the patient as high risk of SA ARD, or other than high risk of SA ARD, based on the presence or absence of SA ARD on D1, the determination of whether the expression levels of the one or more endothelial biomarkers are greater than the one or more pre-determined cut-off expression level, and/or the determination of whether the PaO₂ to FIO₂ ratio is less than a pre-determined cut-off PaO₂ to FIO₂ ratio.

In some embodiments, a classification other than high risk includes a classification of low risk or intermediate risk. In some embodiments, high risk of SA ARD by day 3 of septic shock or other than high risk of SA ARD on day 3 of septic shock is determined.

In some embodiments, a classification of high risk of SA ARD includes: a) a presence of day 1 (D1) SA ARD, and a reduced PaO₂ to FIO₂ ratio; b) a presence of day 1 (D1) SA ARD, a reduced PaO₂ to FIO₂ ratio, and an elevated level of sTM; or c) a presence of day 1 (D1) SA ARD, a non-reduced PaO₂ to FIO₂ ratio, an elevated level of sTM, and a non-elevated level of VCAM-1; and a classification of other than high risk of SA ARD includes: d) an absence of D1 SA ARD; or e) a presence of day 1 (D1) SA ARD, a non-reduced PaO₂ to FIO₂ ratio, a non-elevated level of sTM, and an elevated level of VCAM-1.

In some embodiments, biomarker expression levels can include serum protein biomarker concentrations. In some embodiments, biomarker expression levels can be determined by quantifying serum protein biomarker concentrations and/or by cycle threshold (CT) values.

In some embodiments, the determined biomarker expression levels include expression levels of STM and/or VCAM-1. In some embodiments, the determined biomarker expression levels include expression levels of sTM and VCAM-1. In some embodiments, biomarker levels are determined by serum protein biomarker concentration, and: a) an elevated level of sTM corresponds to a serum sTM concentration greater than about 15.37×103 pg/ml; and b) an elevated level of VCAM-1 corresponds to a serum VCAM-1 concentration greater than about 4.74×106 pg/ml.

In some embodiments, the determination of whether the levels of the one or more biomarkers are non-elevated above a cut-off level includes applying the biomarker expression level data to a decision tree including the two or more biomarkers. In some embodiments, the determination includes application of the decision tree of FIG. 11.

In some embodiments, SA ARD includes direct lung injury and/or indirect lung injury. In some embodiments, SA ARD further includes cardiovascular, respiratory, renal, hepatic, hematologic, and/or neurologic dysfunction, and/or systemic inflammation and/or microvascular endothelial dysfunction, and/or low or no urine output, fluid overload with edema, increased need for supplemental oxygen or intubation and mechanical ventilation, need for dialysis, low blood pressure, multi-organ failure and/or death. In some embodiments, the patient can be, or can have been, undergoing continuous renal replacement therapy (CRRT).

In some embodiments, the classification can be combined with one or more patient demographic data and/or clinical characteristics and/or results from other tests or indicia of septic shock and/or one or more additional biomarkers and/or platelet count. In some embodiments, the one or more additional biomarkers is selected from the group consisting of: interleukin-8 (IL-8), heat shock protein 70 kDa 1B (HSPA1B), C-C Chemokine ligand 3 (CCL3), C-C Chemokine ligand 4 (CCL4), Granzyme B (GZMB), Interleukin-1 α (IL-1a), Matrix metallopeptidase 8 (MMP8), Angiopoietin-1 (Angpt-1), Angiopoietin-1 (Angpt-2), tyrosine kinase with immunoglobulin-like loops and epidermal growth factor homology domains 2 (Tie-2), Inter-Cellular Adhesion Molecule-1 (ICAM-1), P-selectin, E-selectin, and Platelet endothelial cell adhesion molecule-1 (PECAM-1). In some embodiments, the one or more additional biomarkers can be selected from the group consisting of: interleukin-8 (IL-8), heat shock protein 70 kDa 1B (HSPA1B), and C-C Chemokine ligand 3 (CCL3). In some embodiments, the patient demographic data and/or clinical characteristics and/or results from other tests or indicia of septic shock include at least one selected from the group consisting of: the septic shock causative organism, the presence or absence or chronic disease, and/or the age, gender, race, ethnicity, and/or co-morbidities of the patient. In some embodiments, the classification can be combined with one or more additional population-based risk scores. In some embodiments, the one or more population-based risk scores can include at least one selected from the group consisting of: Pediatric Sepsis Biomarker Risk Model (PERSEVERE), Pediatric Sepsis Biomarker Risk Model II (PERSEVERE II), Pediatric Risk of Mortality (PRISM), PRISM III, Pediatric Index of Mortality (PIM), and Pediatric Logistic Organ Dysfunction (PELOD). In some embodiments, the one or more population-based risk scores includes PERSEVERE II.

In some embodiments, the sample can be obtained within the first hour of presentation with septic shock. In some embodiments, the sample can be obtained within the first 24 hours of presentation with septic shock.

In some embodiments, the methods further include administering a treatment including a therapy targeting endothelial dysfunction to a patient that is classified as high risk of SA ARD; or administering a treatment including a corticosteroid to a patient that is classified as other than high risk of SA ARD. In some embodiments, the therapy targeting endothelial dysfunction includes recombinant human sTM and/or one or more Angiopoietin-2 inhibitor. In some embodiments, the methods further include administering a treatment including one or more high risk therapy to a patient that is classified as high risk, or administering a treatment excluding a high risk therapy to a patient that is not high risk, to provide a method of treating a pediatric patient with septic shock. In some embodiments, the one or more high risk therapy includes at least one selected from the group consisting of: biological and/or immune enhancing therapy, extracorporeal membrane oxygenation/life support, plasmapheresis, pulmonary artery catheterization, high volume continuous hemofiltration, non-corticosteroid therapy, adjuvant hemoperfusion, and/or plasma filtration and/or adsorption therapies. In some embodiments, the biological and/or immune enhancing therapy includes administration of recombinant human thrombomodulin, one or more Angiopoietin-2 inhibitor, and/or one or more Tie-2 agonist.

In some embodiments, the patient is enrolled in a clinical trial. In some embodiments, the patient is classified as high risk. In some embodiments, the method includes prognostic enrichment through enrollment of the high risk patient in the clinical trial. In some embodiments, the methods further include administering a treatment including one or more high risk therapy to the patient in the clinical trial.

In some embodiments, the methods further include improving an outcome in a pediatric patient with septic shock.

In some embodiments, the methods further include: analyzing a second dataset associated with a second sample obtained from the treated patient at a second time point, wherein the dataset includes biomarker expression levels of one or more endothelial biomarkers including sTM and VCAM-1 and further includes presence or absence of SA ARD on day 1 (D1) and PaO₂ to FIO₂ ratio for the patient; determining whether the biomarker expression levels of one or more biomarkers are greater than one or more pre-determined cut-off biomarker expression level, and determining whether the PaO₂ to FIO₂ ratio is less than a pre-determined cut-off PaO₂ to FIO₂ ratio; classifying the patient as high risk of SA ARD, or other than high risk of SA ARD, based on the determination of whether the expression levels of each of the biomarkers are greater than the one or more pre-determined cut-off expression level, and/or the determination of whether the PaO₂ to FIO₂ ratio is less than a pre-determined cut-off PaO₂ to FIO₂ ratio; and maintaining the treatment being administered if the patient's high risk classification has not changed, or changing the treatment being administered if the patient's high risk classification has changed.

In some embodiments, the second time point is at least 18 hours after the first time point. In some embodiments, the second time point is in the range of 24 to 96 hours, or longer, after the first time point. In some embodiments, the second time point is about 1 day, 2 days, 3 days, or longer, after the first time point. In some embodiments, the second time point is about 2 days after the first time point. In some embodiments, the first time point is at day 1, wherein day 1 is within 24 hours of a septic shock diagnosis, and the second time point is at day 3.

In some embodiments, a patient classified as high risk after the second time point is administered one or more high risk therapy. In some embodiments, the one or more high risk therapy includes at least one selected from the group consisting of: biological and/or immune enhancing therapy, extracorporeal membrane oxygenation/life support, plasmapheresis, pulmonary artery catheterization, high volume continuous hemofiltration, adjuvant hemoperfusion, and/or plasma filtration and/or adsorption therapies. In some embodiments, the one or more high risk therapy includes a biological and/or immune enhancing therapy. In some embodiments, a patient not classified as high risk after the second time point is administered a treatment excluding a high risk therapy. In some embodiments, a patient not classified as high risk after the second time point is administered a treatment including a corticosteroid. In some embodiments, a patient not classified as high risk after the second time point is administered a treatment excluding a high risk therapy. In some embodiments, the patient classified as high risk and administered one or more high risk therapy after the first time point is not classified as high risk after the second time point.

In some embodiments, one or more biomarker cut-off level is determined by one or more trained machine learning models based on a dataset generated from a cohort of pediatric patients with and without SA ARD. In some embodiments, the data from the cohort of pediatric patients with and without SA ARD is provided to one or more machine learning models as input, and the one or more trained machine learning model is based on a dataset generated from the biomarker cutoff levels in the patients of the cohort. In some embodiments, one or more biomarker cut-off level is determined by a trained machine learning model, and one or more machine learning models is used to classify the patient as high risk of SA ARD, or other than high risk of SA ARD.

In some embodiments, the methods can be performed or used as part of a companion diagnostic or a point of care device or kit.

Further embodiments of the disclosure include diagnostic kits, tests, or arrays including a reporter hybridization probe, and a capture hybridization probe specific for each of two or more mRNA, DNA, or protein biomarkers including sTM and VCAM-1. In some embodiments, the biomarkers further include one or more of interleukin-8 (IL-8), heat shock protein 70 kDa 1B (HSPA1B), C-C Chemokine ligand 3 (CCL3), C-C Chemokine ligand 4 (CCL4), Granzyme B (GZMB), Interleukin-1 α (IL-1a), Matrix metallopeptidase 8 (MMP8), Angiopoietin-1 (Angpt-1), Angiopoietin-1 (Angpt-2), tyrosine kinase with immunoglobulin-like loops and epidermal growth factor homology domains 2 (Tie-2), Inter-Cellular Adhesion Molecule-1 (ICAM-1), P-selectin, E-selectin, and Platelet endothelial cell adhesion molecule-1 (PECAM-1). In some embodiments, the diagnostic kits, tests, or arrays further include a collection cartridge for immobilization of the hybridization probes. In some embodiments, the reporter and the capture hybridization probes include signal and barcode elements, respectively.

Further embodiments of the disclosure include apparatuses or processing devices suitable for detecting two or more biomarkers including sTM and/or VCAM-1. In some embodiments, the biomarkers further include one or more of interleukin-8 (IL-8), heat shock protein 70 kDa 1B (HSPA1B), C-C Chemokine ligand 3 (CCL3), C-C Chemokine ligand 4 (CCL4), Granzyme B (GZMB), Interleukin-1 α (IL-1a), Matrix metallopeptidase 8 (MMP8), Angiopoietin-1 (Angpt-1), Angiopoietin-1 (Angpt-2), tyrosine kinase with immunoglobulin-like loops and epidermal growth factor homology domains 2 (Tie-2), Inter-Cellular Adhesion Molecule-1 (ICAM-1), P-selectin, E-selectin, and Platelet endothelial cell adhesion molecule-1 (PECAM-1).

Further embodiments of the disclosure include compositions including a reporter hybridization probe, and a capture hybridization probe specific for each of two or more biomarkers including sTM and VCAM-1. In some embodiments, the biomarkers further include one or more of interleukin-8 (IL-8), heat shock protein 70 kDa 1B (HSPA1B), C-C Chemokine ligand 3 (CCL3), C-C Chemokine ligand 4 (CCL4), Granzyme B (GZMB), Interleukin-1 α (IL-1a), Matrix metallopeptidase 8 (MMP8), Angiopoietin-1 (Angpt-1), Angiopoietin-1 (Angpt-2), tyrosine kinase with immunoglobulin-like loops and epidermal growth factor homology domains 2 (Tie-2), Inter-Cellular Adhesion Molecule-1 (ICAM-1), P-selectin, E-selectin, and Platelet endothelial cell adhesion molecule-1 (PECAM-1).

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. A block diagram illustrating a computer system 100 upon which embodiments of the present teachings may be implemented. In various embodiments of the present teachings, computer system 100 can include a bus 102 or other communication mechanism for communicating information and a processor 104 coupled with bus 102 for processing information. In various embodiments, computer system 100 can also include a memory, which can be a random-access memory (RAM) 106 or other dynamic storage device, coupled to bus 102 for determining instructions to be executed by processor 104. Memory can also be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

FIG. 2. Flow diagram demonstrating inclusion and exclusion of patients in the derivation cohort.

FIG. 3. Diagram showing overview of study procedures. The derivation cohort (n=625) comprised a prospective multicenter observational study of pediatric patients with septic shock. The test cohort (n=162) comprised data from a prospective single-center study of pediatric patients with sepsis. The derivation cohort was split in a 60:40 ratio into training and holdout validation sets. Stochastic gradient boosting using TreeNet and classification and regression tree (CART) analyses were used in the training set. The performance of the clinical and endothelial biomarker CART model derived then was tested in the holdout validation dataset and test cohorts.

FIG. 4. Relative variable importance shown in TreeNet® models inclusive only of clinical variables.

FIG. 5. Relative variable importance shown in TreeNet® models inclusive of clinical variables and select endothelial biomarkers.

FIG. 6. Area under the receiver operating characteristic curve (AUROC) and weighted misclassification chart for TreeNet® Model inclusive of clinical variables and select endothelial biomarkers predictive of day 3 sepsis associated acute respiratory dysfunction (D3 SA-ARD) in the training dataset (n=375).

FIG. 7. Predictor partial dependence plots between concentrations in pg/ml of select endothelial biomarkers sTM, VCAM-1, and Angpt-1 on the x-axis and fitted half log odds of D3 SA-ARD, the latter shown on the y-axis.

FIG. 8. Surface plot showing interaction between soluble thrombomodulin (sTM) and vascular cell adhesion molecule-1 (VCAM-1) on log odds of persistent sepsis associated acute respiratory dysfunction (D3 SA-ARD) among critically ill children in the training dataset. Patients with high sTM and VCAM-1 concentrations had higher odds of D3 SA-ARD. However, a relatively small subset of patients with low sTM concentrations and had higher VCAM-1 concentrations had a lower odds of D3 SA-ARD showing an interaction between these biomarkers.

FIG. 9. Model summary and confusion matrix of TreeNet® Model inclusive of only clinical variables including age, PRISM-III score, presence of sepsis associated acute respiratory dysfunction on day 1 (D1 SA-ARD) and PaO₂ to FIO₂ ratio <250 to predict risk of day 3 sepsis associated acute respiratory dysfunction (D3 SA-ARD) in the training dataset (n=375).

FIG. 10. Model summary and confusion matrix of TreeNet® Model inclusive of clinical and endothelial biomarkers to predict risk of day 3 sepsis associated acute respiratory dysfunction (D3 SA-ARD) in the training dataset (n=375).

FIG. 11. Classification and regression tree showing the clinicoendothelial biomarker model to stratify patients based on risk of day 3 sepsis-associated acute respiratory dysfunction (SA ARD). The root node at the top of the tree consisted of all patients in the training dataset, of whom 219 patients had day 3 SA ARD. The top decision point was based on the presence of SA ARD on day 1. Patients who did not have day 1 SA ARD were unlikely to have day 3 SA ARD (terminal node 1, 11.6%). Patients with day 1 SA ARD and PaO₂ to FIO₂ ratio of <250 had a high rate of day 3 SA ARD (terminal node 5, 94.9%). Among patients who had day 1 SA ARD but a PaO₂ to FIO₂ ratio of >250, all patients with sTM concentrations of >15,374 pg/mL had day 3 SA ARD (terminal node 4, 100%). Among patients with STM concentrations of #15,374 pg/mL, patients with VCAM-1 concentrations of <4.7×10⁶ pg/mL showed a higher rate of day 3 SA ARD (terminal node 2, 75.7%). In contrast, those with VCAM-1 concentrations of >4.7×10⁶ pg/mL showed a relatively low rate of day 3 SA ARD (terminal node 3, 35.3%). Patients assigned to terminal nodes 1 and 3 were considered to belong to low-risk strata and those assigned to terminal nodes 2, 4, and 5 were considered to belong to high-risk strata. SA ARD=sepsis-associated acute respiratory dysfunction; sTM=soluble thrombomodulin; VCAM-1=vascular cell adhesion molecule 1.

FIG. 12. Graph showing area under the receiver operating characteristic curve for the classification and regression tree model predictive of day 3 sepsis-associated acute respiratory dysfunction in the training dataset (lighter line beginning at further left, 0.88) and on 10-fold cross-validation (darker line, 0.83).

FIG. 13. A rea under the receiver operating characteristic curve (AUROC) of classification and regression tree (CART) model predictive of D3 sepsis associated acute respiratory dysfunction on day 3 (D3 SA-ARD) in the hold-out validation dataset (n=250).

FIG. 14. Classification and regression tree (CART) showing number of patients with and without sepsis associated acute respiratory dysfunction at timepoint 2 (T2 SA-ARD) according to terminal nodes in the test dataset (n=162).

FIG. 15. Area under the receiver operating characteristic curve (AUROC) of classification and regression tree (CART) model predictive of persistent sepsis associated acute respiratory dysfunction at time point 2 (T2 SA-ARD) the test dataset (n=162).

### DETAILED DESCRIPTION

This specification describes various exemplary embodiments of identification and validation of clinically relevant, quantifiable biomarkers associated with sepsis and septic shock, as well as uses thereof. Particular embodiments relate to integration of clinical and endothelial biomarkers for risk prediction in pediatric patients with sepsis-associated acute respiratory dysfunction using a risk model derived and developed as described herein. The disclosure, however, is not limited to these exemplary embodiments and applications or to the manner in which the exemplary embodiments and applications operate or are described herein.

In the specification, reference is made to the accompanying drawings, which form a part hereof. In the specification and drawings, similar symbols typically identify similar components, unless context dictates otherwise. The illustrative embodiments described in the specification and Appendix are not meant to be limiting. Other embodiments may be utilized, and other changes may be made, without departing from the spirit or scope of the subject matter presented herein. It will be readily understood that the aspects of the specification and Appendix, as generally described herein, can be arranged, substituted, combined, separated, and designed in a wide variety of different configurations, all of which are explicitly contemplated herein.

The following description of various embodiments is exemplary and explanatory only and is not to be construed as limiting or restrictive in any way. Other embodiments, features, objects, and advantages of the present teachings will be apparent from the description and accompanying drawings, and from the claims.

The disclosure herein uses affirmative language to describe the numerous embodiments. The disclosure also includes embodiments in which subject matter is excluded, in full or in part, such as substances or materials, method steps and conditions, protocols, or procedures.

It should be understood that any use of subheadings herein are for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular.

### Overview

Endothelial dysfunction is a key feature of sepsis-associated acute respiratory distress syndrome (ARDS) pathophysiologic characteristics, contributing to lung injury in sepsis. Incorporating endothelial biomarkers into risk models may enhance prediction of those with persistent acute respiratory dysfunction.

As described herein, an integrated clinical and endothelial biomarker model has been developed to predict persistent sepsis associated respiratory dysfunction among critically ill children. This model can facilitate prognostic enrichment in future clinical trials of patients and potential to guide therapeutic interventions.

A multi-center derivation cohort and single-center test cohort of prospectively enrolled children with sepsis were established. The derivation cohort was split into training and holdout validation sets. The association between presence of day 3 sepsis-associated acute respiratory dysfunction (D3 SA ARD) and clinical outcomes was established. TreeNet® and classification and regression tree (CART) models were trained using clinical and endothelial biomarkers measured on day 1 of septic shock to predict risk of sepsis-associated acute respiratory dysfunction (SA ARD) on day 3 (D3). The performance of the CART model was tested in the holdout validation set and in the test cohort. Model calibration was tested using Brier scores.

In derivation (n=625) and test cohorts (n=162), children with day 3 sepsis-associated acute respiratory dysfunction (D3 SA ARD) had worse clinical outcomes, including increased mortality, length of mechanical ventilation, and PICU length of stay, as compared with those without. The TreeNet® and CART models yielded comparable results. The variables included in the final CART model were presence of SA ARD on day 1, PaO₂ to FIO₂ ratio of <250, and concentrations of soluble thrombomodulin (sTM) and vascular cell adhesion molecule 1 (VCAM-1). This model showed an area under the receiver operating characteristic curve (AUC) of 0.88 in the training dataset, sensitivity of 0.91 (0.86-0.94), specificity of 0.76 (0.68-0.82), and demonstrated reproducibility in the validation dataset and the test cohort (AUC range, 0.78-0.83). The model was well calibrated with Brier scores ranging from 0.14-0.19.

The decision tree developed herein, which was shown to be predictive of D3 SA-ARD, had an AUROC of 0.96 while the CART model incorporating presence of SA-ARD on day 1, PaO₂/FiO₂ ratio <250, soluble thrombomodulin, and VCAM-1 concentrations had an AUROC of 0.88 in the training dataset. Thus, an effective predictive model incorporating clinical and endothelial biomarkers has been derived and validated for identifying pediatric septic shock patients at high risk for persistent respiratory dysfunction, and, in particular embodiments, to facilitate the early identification of such patients. This is important and highly clinically relevant, given that persistent sepsis-associated respiratory dysfunction-is associated with considerable mortality. The model developed herein, via this integrated approach, can facilitate enrichment of patients in clinical trials and targeted therapeutic interventions that seek to mitigate the risk of SA ARD.

### Definitions of Terms

Unless otherwise noted, terms are to be understood according to conventional usage by those of ordinary skill in the relevant art. For purposes of the present disclosure, the following terms are explained below.

As used herein the specification, “a” or “an” may mean one or more. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in various embodiments.

As used herein, the term “sample” encompasses a sample obtained from a subject or patient. The sample can be of any biological tissue or fluid. Such samples include, but are not limited to, sputum, saliva, buccal sample, oral sample, blood, serum, mucus, plasma, urine, blood cells (e.g., white cells), circulating cells (e.g. stem cells or endothelial cells in the blood), tissue, core or fine needle biopsy samples, cell-containing body fluids, free floating nucleic acids, urine, stool, peritoneal fluid, and pleural fluid, tear fluid, or cells therefrom. Samples can also include sections of tissues such as frozen or fixed sections taken for histological purposes or micro-dissected cells or extracellular parts thereof. A sample to be analyzed can be tissue material from a tissue biopsy obtained by aspiration or punch, excision or by any other surgical method leading to biopsy or resected cellular material. Such a sample can comprise cells obtained from a subject or patient. In some embodiments, the sample is a body fluid that include, for example, blood fluids, serum, mucus, plasma, lymph, ascitic fluids, gynecological fluids, or urine but not limited to these fluids. In some embodiments, the sample can be a non-invasive sample, such as, for example, a saline swish, a buccal scrape, a buccal swab, and the like.

As used herein, “blood” can include, for example, plasma, serum, whole blood, blood lysates, and the like.

As used herein, the term “assessing” includes any form of measurement, and includes determining if an element is present or not. The terms “determining,” “measuring,” “evaluating,” “assessing” and “assaying” can be used interchangeably and can include quantitative and/or qualitative determinations.

As used herein, the term “monitoring” with reference to septic shock refers to a method or process of determining the severity or degree of septic shock or stratifying septic shock based on risk and/or probability of mortality. In some embodiments, monitoring relates to a method or process of determining the therapeutic efficacy of a treatment being administered to a patient.

As used herein, “outcome” can refer to an outcome studied. In some embodiments in accordance with the present disclosure, “outcome” can refer to the presence of severe SA ARD on day 3 of septic shock.

In some embodiments, “outcome” can include survival/mortality at day 7 and/or day 28. The importance of survival/mortality in the context of pediatric septic shock is readily evident. The common choice of 28 days was based on the fact that 28-day mortality is a standard primary endpoint for interventional clinical trials involving critically ill patients. In some embodiments, an increased risk for a poor outcome indicates that a therapy has had a poor efficacy, and a reduced risk for a poor outcome indicates that a therapy has had a good efficacy. In some embodiments, “outcome” can refer to resolution of organ failure after 14 days or 28 days or limb loss. Although mortality/survival is obviously an important outcome, survivors have clinically relevant short- and long-term morbidities that impact quality of life, which are not captured by the dichotomy of “alive” or “dead.” In the absence of a formal, validated quality of life measurement tool for survivors of pediatric septic shock, resolution of organ failure can be used as a secondary outcome measure. For example, the presence or absence of new organ failure over one or more timeframes can be tracked. Patients having organ failure beyond 28 days are likely to survive with significant morbidities having negative consequences for quality of life. Organ failure is generally defined based on published and well-accepted criteria for the pediatric population. Specifically, cardiovascular, respiratory, renal, hepatic, hematologic, and neurologic failure can be tracked. In addition, limb loss can be tracked as a secondary outcome. Although limb loss is not a true “organ failure,” it is an important consequence of pediatric septic shock with obvious impact on quality of life.

In some embodiments, “outcome” can refer to organ dysfunction and/or death after septic shock. In some embodiments, “outcome” can refer to two or more organ dysfunctions or death by day 7 of septic shock. In some embodiments, “outcome” can refer to day 7 cardiovascular, respiratory, renal, hepatic, hematologic, and neurologic dysfunction.

In some embodiments, “outcome” can include complicated course. Complicated course as defined herein relates to persistence of two or more organ failures at day seven of septic shock or 28-day mortality; this can be a composite of death during the study period or the persistence of 2 or more organ dysfunctions on day 7 of septic shock, PICU length of stay (LOS), and/or PIC.

As used herein, the terms “predicting outcome” and “outcome risk stratification” with reference to septic shock refers to a method or process of prognosticating a patient's risk of a certain outcome. In some embodiments, predicting an outcome relates to monitoring the therapeutic efficacy of a treatment being administered to a patient. In some embodiments, predicting an outcome relates to determining a relative risk of an adverse outcome (e.g. complicated course) and/or mortality. In some embodiments, the predicted outcome is associated with administration of a particular treatment or treatment regimen. Such adverse outcome risk and/or mortality can be high risk, moderate risk, moderate-high risk, moderate-low risk, or low risk. Alternatively, such adverse outcome risk can be described simply as high risk or low risk, corresponding to high risk of adverse outcome (e.g. complicated course) and/or mortality probability, or high likelihood of therapeutic effectiveness, respectively. In some embodiments of the present invention, adverse outcome risk can be determined via the biomarker-based SA ARD risk stratification as described herein. In some embodiments, predicting an outcome relates to determining a relative risk of SA ARD. Such mortality risk can be high risk, moderate risk, moderate-high risk, moderate-low risk, or low risk. Alternatively, such mortality risk can be described simply as high risk or low risk, corresponding to high risk of death or high likelihood of survival, respectively. As related to the terminal nodes of the decision trees described herein, a “high risk terminal node” corresponds to an increased probability of adverse outcome (e.g. complicated course) and/or mortality according to a particular treatment or treatment regimen, whereas a “low risk terminal node” corresponds to a decreased probability of adverse outcome (e.g. complicated course) and/or mortality according to a particular treatment or treatment regimen.

As used herein, the term “high risk clinical trial” refers to one in which the test agent has “more than minimal risk” (as defined by the terminology used by institutional review boards, or IRBs). In some embodiments, a high risk clinical trial is a drug trial.

As used herein, the term “low risk clinical trial” refers to one in which the test agent has “minimal risk” (as defined by the terminology used by IRBs). In some embodiments, a low risk clinical trial is one that is not a drug trial. In some embodiments, a low risk clinical trial is one that that involves the use of a monitor or clinical practice process. In some embodiments, a low risk clinical trial is an observational clinical trial.

As used herein, the terms “modulated” or “modulation,” or “regulated” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

As used herein, the term “subject” refers to any member of the animal kingdom. In some embodiments, a subject is a human patient. In some embodiments, a subject is a pediatric patient. In some embodiments, a pediatric patient is a patient under 18 years of age, while an adult patient is 18 or older. Unless stated otherwise, the terms “patient” or “child” (or “patients” or “children”) refer to a pediatric patient (i.e., under 18 years old).

As used herein, the terms “treatment,” “treating,” “treat,” and the like, with respect to a disease or condition, can refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. For example, a treatment can include executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

“Treatment,” as used herein, thus can cover any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” can also encompass delivery of an agent or administration of a therapy in order to provide for a pharmacologic effect, even in the absence of a disease or condition.

The term “therapeutically effective” or “therapeutically effective amount” as used throughout this application can refer to an amount effective to achieve a desired and/or beneficial effect, and/or anything that promotes or enhances the well-being of the subject with respect to the medical treatment of a condition. This includes, but is not limited to, a reduction in the frequency or severity of one or more signs or symptoms of a disease. An effective amount can be administered in one or more administrations. In the methods, a therapeutically effective amount is an amount appropriate to treat an indication. By treating an indication is meant achieving any desirable effect, such as one or more of palliate, ameliorate, stabilize, reverse, slow, or delay disease progression, increase the quality of life, or to prolong life. Such achievement can be measured by any suitable method, such as measurement of tumor size or blood cell count, or any other suitable measurement.

The terms “effective amount” or “effective dose” as used herein have their plain and ordinary meaning as understood in light of the specification, and can refer to that amount of a recited composition or compound that, results in an observable effect. Actual dosage levels of active ingredients in an active composition of the presently disclosed subject matter can be varied so as to administer an amount of the active composition or compound that, is effective to achieve the desired response for a particular subject and/or application. The selected dosage level wall depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The term “disease state” as used herein, can generally refer to a condition that affects the structure or function of an organism. Disease states can include, for example, stages of a disease progression.

As used herein, the term “assessing” can include any form of measurement, and includes determining if an element is present or not. The terms “determining,” “measuring,” “evaluating,” “assessing” and “assaying” can be used interchangeably and can include quantitative and/or qualitative determinations.

As used herein, the terms “modulated” or “modulation,” or “regulated” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

As used herein, the term “marker” or “biomarker” can refer to any measurable substance taken as a sample from a subject whose presence is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a disease state, a health state, an asymptomatic state, a symptomatic state, etc.). The term “biomarker” may be used interchangeably with the term “marker”. The term “marker” or “biomarker” can include a biological molecule, such as, for example, a nucleic acid, peptide, protein, hormone, and the like, whose presence or concentration can be detected and correlated with a known condition, such as a disease state. It can also be used to refer to a differentially expressed gene whose expression pattern can be utilized as part of a predictive, prognostic or diagnostic process in healthy conditions or a disease state, or which, alternatively, can be used in methods for identifying a useful treatment or prevention therapy.

As used herein, the term “expression levels” refers, for example, to a determined level of biomarker expression. The term “pattern of expression levels” refers to a determined level of biomarker expression compared either to a reference (e.g. a housekeeping gene or inversely regulated genes, or other reference biomarker) or to a computed average expression value (e.g. in DNA-chip analyses). A pattern is not limited to the comparison of two biomarkers but is more related to multiple comparisons of biomarkers to reference biomarkers or samples. A certain “pattern of expression levels” can also result and be determined by comparison and measurement of several biomarkers as disclosed herein and display the relative abundance of these transcripts to each other.

As used herein, a “reference pattern of expression levels” refers to any pattern of expression levels that can be used for the comparison to another pattern of expression levels. In some embodiments of the invention, a reference pattern of expression levels is, for example, an average pattern of expression levels observed in a group of healthy or diseased individuals, serving as a reference group.

As used herein, the term “decision tree” refers to a standard machine learning technique for multivariate data analysis and classification. Decision trees can be used to derive easily interpretable and intuitive rules for decision support systems.

As used herein, the term “cellular phenotype” can refer to any determinable, observable, and/or measurable characteristic associated with a cell population.

As used herein, a “model” can include one or more in vitro or in vivo disease models; a model can also include algorithms, one or more mathematical techniques, one or more machine learning algorithms, or a combination thereof. A model can be used in a process and/or applied to an assay, in accordance with various embodiments as disclosed herein.

As used herein, a “process” can include one or more steps involving one or more features of one or more model as disclosed herein.

The terms “function” and “functional” as used herein have their plain and ordinary meaning as understood in light of the specification, and can refer to a biological, enzymatic, or therapeutic function.

All references cited herein are incorporated by reference in their entirety. Also incorporated herein by reference in their entirety include: U.S. Patent Application No. 61/595,996, BIOMARKERS OF SEPTIC SHOCK, filed on Feb. 7, 2012; U.S. Provisional Application No. 61/721,705, A MULTI-BIOMARKER-BASED OUTCOME RISK STRATIFICATION MODEL FOR ADULT SEPTIC SHOCK, filed on Nov. 2, 2012; International Patent Application No. PCT/US2013/25223, A MULTI-BIOMARKER-BASED OUTCOME RISK STRATIFICATION MODEL FOR PEDIATRIC SEPTIC SHOCK, filed on Feb. 7, 2013; International Patent Application No. PCT/US2013/25221, A MULTI-BIOMARKER-BASED OUTCOME RISK STRATIFICATION MODEL FOR ADULT SEPTIC SHOCK, filed on Feb. 7, 2013; U.S. Provisional Application No. 61/908,613, TEMPORAL PEDIATRIC SEPSIS BIOMARKER RISK MODEL, filed on Nov. 25, 2013; International Patent Application No. PCT/US2014/067438, TEMPORAL PEDIATRIC SEPSIS BIOMARKER RISK MODEL, filed on Nov. 25, 2014; U.S. patent application Ser. No. 15/998,427, SEPTIC SHOCK ENDOTY PING STRATEGY AND MORTALITY RISK FOR CLINICAL APPLICATION, filed on Aug. 15, 2018; U.S. Provisional Application No. 62/616,646, TEMPORAL ENDOTYPE TRANSITIONS REFLECT CHANGING RISK AND TREATMENT RESPONSE IN PEDIATRIC SEPTIC SHOCK, filed on Jan. 12, 2018; International Application No. PCT/US2017/032538, SIMPLIFICATION OF A SEPTIC SHOCK ENDOTY PING STRATEGY FOR CLINICAL APPLICATIONS, filed on May 12, 2017; U.S. Provisional Application No. 62/335,803, SIMPLIFICATION OF A SEPTIC SHOCK ENDOTYPING STRATEGY FOR CLINICAL APPLICATIONS, filed on May 13, 2016; U.S. Provisional Application No. 62/427,778, SIMPLIFICATION OF A SEPTIC SHOCK ENDOTYPING STRATEGY FOR CLINICAL APPLICATIONS, filed on Nov. 29, 2016; U.S. Provisional Application No. 62/428,451, SIMPLIFICATION OF A SEPTIC SHOCK ENDOTYPING STRATEGY FOR CLINICAL APPLICATIONS, filed on Nov. 30, 2016; U.S. Provisional Application No. 62/446,216, SIMPLIFICATION OF A SEPTIC SHOCK ENDOTYPING STRATEGY FOR CLINICAL APPLICATIONS, filed on Jan. 13, 2017; U.S. patent application Ser. No. 16/539,128, SEPTIC SHOCK ENDOTYPING STRATEGY AND MORTALITY RISK FOR CLINICAL APPLICATION, filed on Aug. 13, 2019; U.S. Provisional Application No. 62/764,831, Endotype Transitions During the A cute Phase of Pediatric Septic Shock Reflect Changing Risk and Treatment Response, filed on Aug. 15, 2018; U.S. Provisional Application No. 63/149,744, A CONTINUOUS METRIC TO ASSESS THE INTERACTION BETWEEN ENDOTY PE ASSIGNMENT AND CORTICOSTEROID RESPONSIVENESS IN SEPTIC SHOCK, filed on Feb. 16, 2021; International Patent Application No. PCT/US2022/016642, A CONTINUOUS METRIC TO ASSESS THE INTERACTION BETWEEN ENDOTYPE ASSIGNMENT AND CORTICOSTEROID RESPONSIVENESS IN SEPTIC SHOCK, filed on Feb. 16, 2022; U.S. Provisional Application No. 63/347,504, PREDICTING PERSISTENT MULTIPLE ORGAN DYSFUNCTION IN THE PEDIATRIC POPULATION AFTER CARDIOPULMONARY BY PASS USING SEPSIS PROGNOSTIC BIOMARKERS, filed on May 31, 2022; and U.S. Provisional Patent Application No. PEDIATRIC SEPSIS MULTIPLE ORGAN DYSFUNCTION SYNDROME RISK PREDICTION MODEL, filed on Jun. 1, 2022.

### Sepsis-Associated Acute Respiratory Distress Syndrome

Sepsis is a common cause of ARDS, with severe cases resulting in the death of every 1 in 3 affected children. Moreover, survivors experience significant long-term morbidity, including tracheostomy dependence, frequent rehospitalization, and a reduced quality of life.

Despite this burden of disease, treatment options remain limited to antibiotics, lung protective ventilation, and organ support. Yet, clinical and biological heterogeneity among patients adds considerable complexity, underscoring the need to identify patients at high risk of persistent sepsis-associated acute respiratory dysfunction (SA ARD) who may benefit from targeted intervention(s). Y et, attempts to develop reliable risk stratification tools for pediatric patients have been limited by sample size.

To date, research on clinical variables and candidate biomarkers, including those reflective of immune, epithelial, and endothelial pathways, to identify patients who are critically ill and at high risk of poor outcomes has been unsuccessful to date. Among these pathways, the present disclosure has found that endothelial dysfunction plays a particularly central role in the pathophysiologic characteristics of sepsis-related organ failure. The endothelium releases cytokines and chemokines, upregulates adhesion molecules that promote leukocyte migration and inflammation, increases permeability leading to tissue hypoperfusion, and disrupts anticoagulant mechanisms, resulting in microthrombi and impaired oxygen delivery during sepsis. Furthermore, genomic studies among adults with SA ARD have implicated genes with key roles in vascular signaling and have identified candidate targets with potentially causal roles in the evolution of disease pathobiology. There is an ongoing need for clinically useful risk stratification models for SA ARD, in pediatric as well as in adult patients.

### Determining Risk of SA ARD

As described herein, the present inventors have derived and validated risk-prediction models incorporating clinical variables and endothelial biomarkers early in the course of illness to reliably identify pediatric patients with sepsis at highest risk of persistent SA ARD, taking into consideration the disproportionate contribution of endothelial dysfunction to SA ARD biology. This study leveraged a large multicenter derivation cohort of critically ill children with septic shock to train models rigorously using supervised machine learning approaches. The performance of the risk model was subsequently validated in a holdout validation dataset and an independent test cohort to demonstrate prognostic usefulness and model reproducibility.

The study and results described herein demonstrated a significant association between persistent SA ARD and poor clinical outcomes in a large cohort of critically ill children with septic shock. Predictive models were developed and validated using clinical data and endothelial biomarkers measured on day 1 of pediatric septic shock to identify reliably patients at risk of persistent SA ARD by day 3. The CART model, which was tested in a holdout validation dataset and an independent cohort, showed prognostic usefulness and reproducibility.

Recent studies have focused on developing risk models predictive of acute hypoxemic respiratory failure among adults with sepsis. These models have included combinations of between 6 and 13 clinical variables, laboratory values, and select biomarkers including IL-6, with AUCs ranging from 0.71 to 0.81.

In contrast, studies predictive of SA ARDS and related outcomes among children have been relatively limited. A validated biomarker-based risk model consisting of patient age, C-C chemokine ligand 3, heat shock protein family A member 1B, and IL-8 to predict mortality-risk in pediatric ARDS has been described to have a model performance evaluated at 3 different time points with an AUC ranging from 0.68 to 0.78.

Few pediatric studies have focused on establishing the association between endothelial biomarkers and risk of SA ARDS outcomes. sTM can predict mortality risk and extrapulmonary organ failure in children with ARDS (RESTORE trial). Also, the present inventors shown that angiopoietin 2 to angiopoietin 1 ratio, VCAM-1, and von Willebrand factor can discriminate between direct and indirect lung injury in pediatric sepsis. Patients with indirect or extrapulmonary sepsis meeting criteria for ARDS had elevated levels of angiopoietin 2, angiopoietin 2 to angiopoietin 1 ratio, von Willebrand factor, and endocan relative to those without ARDS and were associated with higher rates of a complicated course.

In the present study, the inclusion of commonly available clinical measures along with selected biomarkers allowed for the successful identification of patients at risk of persistent SA ARD. The biomarkers added specificity to the model by identifying a subset of patients with less severe day 1 SA ARD (defined by a PaO₂ to FIO₂ ratio of >250) who nevertheless would meet criteria for persistent SA ARD. Such subsets characterized by high sTM concentrations or relatively low sTM concentrations and low VCAM-1 concentrations can represent a potential subgroup for enrichment in clinical trials for interventions aimed at halting disease progression, as underscored by the Pediatric Acute Lung Injury Consensus Conference guidelines.

Among the endothelial biomarkers tested, sTM—a cleavage product from the vascular endothelium, where it serves as a major component of the endogenous anticoagulation system—showed the highest odds of day 3 SA ARD. The risk predictive model described herein can be used to enrich patients in future clinical trials of therapies targeting endothelial dysfunction, such as recombinant human STM and Angiopoietin-2 inhibitors.

Another intriguing finding in the present study is the protective association between VCAM-1 and day 3 SA ARD outcomes. This was particularly evident among patients with relatively low sTM concentrations, demonstrating a complex interaction, which merits further investigation.

### Additional Patient Information

The demographic data, clinical characteristics, and/or results from other tests or indicia of septic shock specific to a pediatric patient with septic shock can affect the patient's outcome risk. Accordingly, such demographic data, clinical characteristics, and/or results from other tests or indicia of septic shock can be incorporated into the methods described herein which allow for stratification of individual pediatric patients in order to determine the patient's outcome risk. Such demographic data, clinical characteristics, and/or results from other tests or indicia of septic shock can also be used in combination with the methods described herein which allow for stratification of individual pediatric patients in order to determine the patient's outcome risk.

Such pediatric patient demographic data can include, for example, the patient's age, race, ethnicity, gender, and the like. In some embodiments, the biomarker-based SA ARD risk stratification described herein can incorporate or be used in combination with the patient's age, race, ethnicity, and/or gender to determine an outcome risk.

Such patient clinical characteristics and/or results from other tests or indicia of septic shock can include, for example, the patient's co-morbidities and/or septic shock causative organism, and the like.

Patient co-morbidities can include, for example, acute lymphocytic leukemia, acute myeloid leukemia, aplastic anemia, atrial and ventricular septal defects, bone marrow transplantation, caustic ingestion, chronic granulomatous disease, chronic hepatic failure, chronic lung disease, chronic lymphopenia, chronic obstructive pulmonary disease (COPD), congestive heart failure (NY HA Class IV CHF), Cri du Chat syndrome, cyclic neutropenia, developmental delay, diabetes, DiGeorge syndrome, Down syndrome, drowning, end stage renal disease, glycogen storage disease type 1, hematologic or metastatic solid organ malignancy, hemophagocytic lymphohistiocytosis, hepatoblastoma, heterotaxy, hydrocephalus, hypoplastic left heart syndrome, IPEX Syndrome, kidney transplant, Langerhans cell histiocytosis, liver and bowel transplant, liver failure, liver transplant, medulloblastoma, metaleukodystrophy, mitochondrial disorder, multiple congenital anomalies, multi-visceral transplant, nephrotic syndrome, neuroblastoma, neuromuscular disorder, obstructed pulmonary veins, Pallister Killian syndrome, Prader-Willi syndrome, requirement for chronic dialysis, requirement for chronic steroids, retinoblastoma, rhabdomyosarcoma, rhabdosarcoma, sarcoma, seizure disorder, severe combined immune deficiency, short gut syndrome, sickle cell disease, sleep apnea, small bowel transplant, subglottic stenosis, tracheal stenosis, traumatic brain injury, trisomy 18, type 1 diabetes mellitus, unspecified brain tumor, unspecified congenital heart disease, unspecified leukemia, VATER Syndrome, Wilms tumor, and the like. Any one or more of the above patient co-morbidities can be indicative of the presence or absence of chronic disease in the patient. Septic shock causative organisms can include, for example, A Cinetobacter baumannii, Adenovirus, Bacteroides species, Candida species, Capnotyophaga jenuni, Cytomegalovirus, Enterobacter cloacae, Enterococcus faecalis, Escherichia coli, Herpes simplex virus, Human metapneumovirus, Influenza A, Klebsiella pneumonia, Micrococcus species, mixed bacterial infection, Moraxella catarrhalis, Neisseria meningitides, Parainfluenza, Pseudomonas species, Serratia marcescens, Staphylococcus aureus, Streptococcus agalactiae, Streptococcus milleri, Streptococcus pneumonia, Streptococcus pyogenes, unspecified gram negative rods, unspecified gram positive cocci, and the like.

In some embodiments, the biomarker-based SA ARD risk stratification as described herein can incorporate the patient's co-morbidities to determine an outcome risk and/or mortality probability. In some embodiments, the biomarker-based SA ARD risk stratification as described herein can incorporate the patient's septic shock causative organism to determine an outcome risk and/or mortality probability.

In some embodiments, the biomarker-based SA ARD risk stratification as described herein can be used in combination with the patient's co-morbidities to determine an outcome risk and/or mortality probability. In some embodiments, the biomarker-based SA ARD risk stratification as described herein can be used in combination with the patient's septic shock causative organism to determine an outcome risk and/or mortality probability.

Certain embodiments of the invention include using quantification data from a gene-expression analysis and/or from a protein, mRNA, and/or DNA analysis, from a sample of blood, urine, saliva, broncho-alveolar lavage fluid, or the like. Embodiments of the invention include not only methods of conducting and interpreting such tests but also include reagents, compositions, kits, tests, arrays, apparatuses, processing devices, assays, and the like, for conducting the tests. The compositions and kits of the present invention can include one or more components which enable detection of the biomarkers disclosed herein and combinations thereof and can include, but are not limited to, primers, probes, cDNA, enzymes, covalently attached reporter molecules, and the like.

Diagnostic-testing procedure performance is commonly described by evaluating control groups to obtain four critical test characteristics, namely positive predictive value (PPV), negative predictive value (NPV), sensitivity, and specificity, which provide information regarding the effectiveness of the test. The PPV of a particular diagnostic test represents the proportion of positive tests in subjects with the condition of interest (i.e. proportion of true positives); for tests with a high PPV, a positive test indicates the presence of the condition in question. The NPV of a particular diagnostic test represents the proportion of negative tests in subjects without the condition of interest (i.e. proportion of true negatives); for tests with a high NPV, a negative test indicates the absence of the condition. Sensitivity represents the proportion of subjects with the condition of interest who will have a positive test; for tests with high sensitivity, a positive test indicates the presence of the condition in question. Specificity represents the proportion of subjects without the condition of interest who will have a negative test; for tests with high specificity, a negative test indicates the absence of the condition.

The threshold for the disease state can alternatively be defined as a 1-D quantitative score, or diagnostic cutoff, based upon receiver operating characteristic (ROC) analysis. The quantitative score based upon ROC analysis can be used to determine the specificity and/or the sensitivity of a given diagnosis based upon subjecting a patient to a decision tree described herein in order to predict an outcome for a pediatric patient with septic shock.

The correlations disclosed herein, between pediatric patient septic shock biomarker levels and/or mRNA levels and/or gene expression levels, and/or protein expression levels, provide a basis for conducting a diagnosis of septic shock, or for conducting a stratification of patients with septic shock, or for enhancing the reliability of a diagnosis of septic shock by combining the results of a quantification of a septic shock biomarker with results from other tests or indicia of septic shock, or for determining an appropriate treatment regimen for a pediatric patient with septic shock. For example, the results of a quantification of one biomarker could be combined with the results of a quantification of one or more additional biomarker, protein, cytokine, mRNA, or the like. Thus, even in situations in which a given biomarker correlates only moderately or weakly with septic shock, providing only a relatively small PPV, NPV, specificity, and/or sensitivity, the correlation can be one indicium, combinable with one or more others that, in combination, provide an enhanced clarity and certainty of diagnosis. Accordingly, the methods and materials of the invention are expressly contemplated to be used both alone and in combination with other tests and indicia, whether quantitative or qualitative in nature.

### PERSEVERE, PERSEVERE II, and Other Population-Based Risk Scores

Other risk models for estimating baseline mortality risk in children with septic shock have been previously derived and validated; none of these models is specific for SA ARD. These include PERSEVERE, which is based on a panel of 12 serum protein biomarkers measured from blood samples obtained during the first 24 hours of a septic shock diagnosis, selected from among 80 genes having an association with mortality risk in pediatric septic shock. Of those 12 serum biomarkers, the derived and validated PERSEVERE model is based on Interleukin-8 (IL-8), Heat shock protein 70 kDA (HSP70), C-C Chemokine ligand 3 (CCL3), C-C Chemokine ligand 4 (CCL4), Granzyme B (GZMB), Interleukin-1 α (IL-1a), and Matrix metallopeptidase 8 (MMP8). PERSEVERE additionally takes patient age into account.

The PERSEVERE decision tree has 8 terminal nodes. Of these, 3 terminal nodes of the PERSEVERE decision tree are determined to be low risk/low mortality probability (terminal nodes 2, 4, and 7), while 5 terminal nodes of the PERSEVERE decision tree are determined to be intermediate to high risk/high mortality probability (terminal nodes 1, 3, 5, 6, and 8). In some embodiments, a low risk/low mortality probability terminal nodes has a mortality probability between 0.000 and 0.025, while an intermediate to high risk/high mortality probability terminal nodes has a mortality probability greater than 0.025.

In some embodiments of the present invention, a patient sample can be analyzed for the PERSEVERE serum protein biomarkers IL-8 and HSP70, as well as for the endothelial biomarkers ICAM-1, Thrombomodulin, A ngpt-2/A ngpt-1, and/or Angpt-2/Tie-2.

In some embodiments of the present invention, the PERSEVERE mortality probability stratification can be used in combination with the biomarker-based SA ARD risk stratification as described herein. In some embodiments, the biomarker-based SA ARD risk stratification, as described herein, can be used in combination with a patient endotyping strategy and/or Z score determination. In some embodiments, the combination of a biomarker-based SA ARD risk stratification, with an endotyping strategy and/or Z score determination, can be used to determine an appropriate treatment regimen for a patient. For example, such combinations can be used to identify which patients are more likely to benefit from corticosteroids. In some embodiments, a patient having a classification of low risk or intermediate risk can be treated with corticosteroids.

PERSEVERE II is another previously derived and validated risk model for estimating baseline mortality risk in children with septic shock and is based on a panel of 5 serum protein biomarkers measured from blood samples obtained during the first 24 hours of a septic shock diagnosis. Of those 5 serum biomarkers, the derived and validated PERSEVERE II model is based on interleukin-8 (IL-8), C-C chemokine ligand 3 (CCL3), and heat shock protein 70 kDa 1B (HSPA1B), as well as platelet count.

The PERSEVERE II decision tree has 5 terminal nodes. Of these, 3 terminal nodes of the PERSEVERE II decision tree are determined to be low risk/low mortality probability (terminal nodes 1, 2, and 4), while 2 terminal nodes of the PERSEVERE II decision tree are determined to be intermediate to high risk/high mortality probability (terminal nodes 3 and 5). In some embodiments, a low risk/low mortality probability terminal nodes has a mortality probability between 0.000 and 0.025, while an intermediate to high risk/high mortality probability terminal nodes has a mortality probability greater than 0.025.

In some embodiments of the present invention, a patient sample can be analyzed for the PERSEVERE II serum protein biomarkers IL-8, CCL3, and HSPA1B, and platelet count, as well as for the endothelial biomarkers Tie-2, Angpt-2, and sTM.

In some embodiments of the present invention, the PERSEVERE II mortality probability stratification can be used in combination with the biomarker-based SA ARD risk stratification as described herein. In some embodiments, the biomarker-based SA ARD risk stratification, as described herein, can be used in combination with a patient endotyping strategy and/or Z score determination. In some embodiments, the combination of a biomarker-based SA ARD risk stratification, with an endotyping strategy and/or Z score determination, can be used to determine an appropriate treatment regimen for a patient. For example, such combinations can be used to identify which patients are more likely to benefit from corticosteroids.

A number of additional models that generate mortality prediction scores based on physiological variables have been developed to date. These can include the PRISM, Pediatric Index of Mortality (PIM), and/pediatric logistic organ dysfunction (PELOD) models, and the like.

Such models can be very effective for estimating population-based outcome risks but are not intended for stratification of individual patients. The methods described herein which allow for stratification of individual patients can be used alone or in combination with one or more existing population-based risk scores.

In some embodiments, the biomarker-based SA ARD risk stratification described herein can be used with one or more additional population-based risk scores. In some embodiments, the biomarker-based SA ARD risk stratification described herein can be used in combination with PRISM. In some embodiments, the biomarker-based SA ARD risk stratification described herein can be used in combination with PIM. In some embodiments, the biomarker-based SA ARD risk stratification herein can be used in combination with PELOD. In some embodiments, the biomarker-based SA ARD risk stratification described herein can be used in combination with a population-based risk score other than PRISM, PIM, and PELOD.

### High Risk Therapies

High risk, invasive therapeutic and support modalities can be used to treat septic shock. The methods described herein which allow for the patient's outcome risk to be determined can help inform clinical decisions regarding the application of high risk therapies to specific pediatric patients, based on the patient's outcome risk.

High risk therapies include, for example, adjuvant hemoperfusion, plasma filtration and adsorption therapies, extracorporeal membrane oxygenation/life support, plasmapheresis, pulmonary artery catheterization, high volume continuous hemofiltration, and the like. High risk therapies can also include non-corticosteroid therapies, e.g. alternative therapies and/or high risk therapies. In particular, patients at high risk of SA ARD can be treated with immune enhancing therapies, such as, for example, recombinant human thrombomodulin, Angiopoietin-2 inhibitors, Tie-2 agonists, and the like.

In some embodiments, individualized treatment can be provided to a pediatric patient by selecting a pediatric patient classified as high risk by the methods described herein for one or more high risk therapies. In some embodiments, individualized treatment can be provided to a pediatric patient by excluding a pediatric patient classified as low risk from one or more high risk therapies.

### Computer Implemented System

In various embodiments, the systems and methods for recognizing home activities by deep learning subtle vibrations on an interior surface of a house from a single point using vibration sensing devices can be implemented via computer software or hardware. Refer to the Appendix for further information regarding the system, devices and methods provided herein, in accordance with various embodiments.

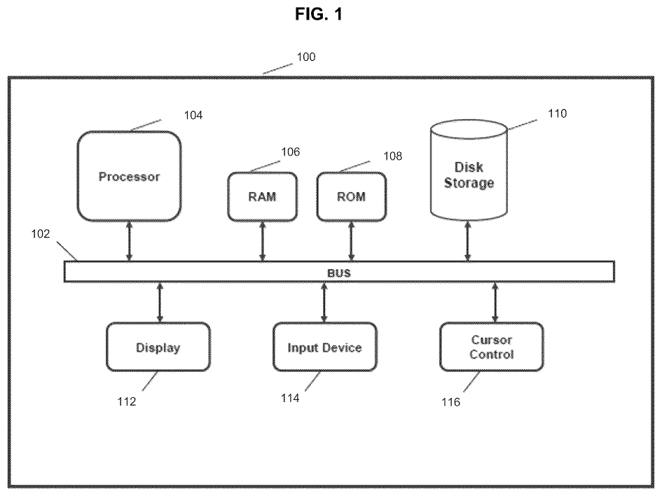

FIG. 1 is a block diagram illustrating a computer system 100 upon which embodiments of the present teachings may be implemented. In various embodiments of the present teachings, computer system 100 can include a bus 102 or other communication mechanism for communicating information and a processor 104 coupled with bus 102 for processing information. In various embodiments, computer system 100 can also include a memory, which can be a random-access memory (RAM) 106 or other dynamic storage device, coupled to bus 102 for determining instructions to be executed by processor 104. Memory can also be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

In various embodiments, computer system 100 can be coupled via bus 102 to a display 112, such as a cathode ray tube (CRT) or liquid crystal display (LCD), for displaying information to a computer user. An input device 114, including alphanumeric and other keys, can be coupled to bus 102 for communication of information and command selections to processor 104. Another type of user input device is a cursor control 116, such as a mouse, a trackball or cursor direction keys for communicating direction information and command selections to processor 104 and for controlling cursor movement on display 112. This input device 114 typically has two degrees of freedom in two axes, a first axis (i.e., x) and a second axis (i.e., y), that allows the device to specify positions in a plane. However, it should be understood that input devices 114 allowing for 3-dimensional (x, y and z) cursor movement are also contemplated herein.

Consistent with certain implementations of the present teachings, results can be provided by computer system 100 in response to processor 104 executing one or more sequences of one or more instructions contained in memory 106. Such instructions can be read into memory 106 from another computer-readable medium or computer-readable storage medium, such as storage device 110. Execution of the sequences of instructions contained in memory 106 can cause processor 104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 104 for execution. Such a medium can take many forms, including but not limited to, non-volatile media, volatile media, and transmission media. Examples of non-volatile media can include, but are not limited to, dynamic memory, such as memory 106. Examples of transmission media can include, but are not limited to, coaxial cables, copper wire, and fiber optics, including the wires that comprise bus 102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 104 of computer system 100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 100 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 100, whereby processor 104 would execute the analyses and determinations provided by these engines, subject to instructions provided by any one of, or a combination of, memory components 106/108/110 and user input provided via input device 114.

In various embodiments, the methods of the present teachings can involve deep learning and/or machine learning and/or one or more neural network, such as a deep neural network, and the like. It should be understood that while deep learning and such processes may be discussed in conjunction with various embodiments herein, the various embodiments herein are not limited to being associated only with deep learning tools. As such, machine learning and/or artificial intelligence tools generally may be applicable as well. Moreover, the terms deep learning, machine learning, and artificial intelligence may even be used interchangeably in generally describing the various embodiments of systems, software and methods herein.

A deep neural network (DNN) generally, such as a convolutional neural network (CNN), generally accomplishes an advanced form of image processing and classification/detection by first looking for low level features such as, for example, edges and curves, and then advancing to more abstract (e.g., unique to the type of images being classified) concepts through a series of convolutional layers. A DNN/CNN can do this by passing an image through a series of convolutional, nonlinear, pooling (or downsampling, as will be discussed in more detail below), and fully connected layers, and get an output. A gain, the output can be a single class or a probability of classes that best describes the image or detects objects on the image.

Regarding layers in a CNN, for example, the first layer is generally a convolutional layer (Conv). This first layer will process the image's representative array using a series of parameters. Rather than processing the image as a whole, a CNN will analyze a collection of image sub-sets using a filter (or neuron or kernel). The sub-sets will include a focal point in the array as well surrounding points. For example, a filter can examine a series of 5×5 areas (or regions) in a 32×32 image. These regions can be referred to as receptive fields. Since the filter must possess the same depth of the input, an image with dimensions of 32×32×3 would have a filter of the same depth (e.g., 5×5×3). The actual step of convolving, using the exemplary dimensions above, would involve sliding the filter along the input image, multiplying filter values with the original pixel values of the image to compute element wise multiplications, and summing these values to arrive at a single number for that examined portion of the image.

After completion of this convolving step, using a 5×5×3 filter, an activation map (or filter map) having dimensions of 28×28×1 will result. For each additional layer used, spatial dimensions are better preserved such that using two filters will result in an activation map of 28×28×2. Each filter will generally have a unique feature it represents (e.g., colors, edges, curves, etc.) that, together, represent the feature identifiers required for the final image output. These filters, when used in combination, allow the CNN to process an image input to detect those features present at each pixel. Therefore, if a filter serves as a curve detector, the convolving of the filter along the image input will produce an array of numbers in the activation map that correspond to high likelihood of a curve (high summed element wise multiplications), low likelihood of a curve (low summed element wise multiplications) or a zero value where the input volume at certain points provided nothing that would activate the curve detector filter. As such, the greater number of filters (also referred to as channels) in the Conv, the more depth (or data) that is provided on the activation map, and therefore more information about the input that will lead to a more accurate output.

Balanced with accuracy of the CNN is the processing time and power needed to produce a result. In other words, the more filters (or channels) used, the more time and processing power needed to execute the Conv. Therefore, the choice and number of filters (or channels) to meet the needs of the CNN method are specifically chosen to produce as accurate an output as possible while considering the time and power available.

To enable further a CNN to detect more complex features, additional Conv layers can be added to analyze what outputs from the previous Conv layer (i.e., activation maps). For example, if a first Conv layers looks for a basic feature such as a curve or an edge, a second Conv layer can look for a more complex feature such as shapes, which can be a combination of individual features detected in an earlier Conv layer. By providing a series of Conv layers, the CNN can detect increasingly higher-level features to arrive eventually at the specific desired object detection. Moreover, as the Conv layers stack on top of each other, analyzing the previous activation map output, each Conv layer in the stack is naturally going to analyze a larger and larger receptive field by virtue of the scaling down that occurs at each Conv level, thereby allowing the CNN to respond to a growing region of pixel space in detecting the object of interest.

A CNN architecture generally consists of a group of processing blocks, including at least one processing block for convoluting an input volume (image) and at least one for deconvolution block (or transpose convolution). Additionally, the processing blocks can include at least one pooling block and unpooling block. Pooling blocks can be used to scale down an image in resolution to produce an output available for Conv. This can provide computational efficiency (efficient time and power), which can in turn improve actual performance of the CNN. Those these pooling, or subsampling, blocks keep filters small and computational requirements reasonable, these blocks coarsen the output (can result in lost spatial information within a receptive field), reducing it from the size of the input by a factor equal to the pixel stride of the receptive fields of the output units.

Unpooling blocks can be used to reconstruct a these coarse outputs to produce an output volume with the same dimensions as the input volume. An unpooling block can be considered a reverse operation of a convoluting block to return an activation output to the original input volume dimension.

However, the unpooling process generally just simply enlarges the coarse outputs into a sparse activation map. To avoid this result, the deconvolution block densifies this sparse activation map to produce both and enlarged and dense activation map that eventually, after any further necessary processing, a final output volume with size and density much closer to the input volume. As a reverse operation of the convolution block, rather than reducing multiple array points in the receptive field to a single number, the deconvolution block associate a single activation output point with a multiple outputs to enlarge and densify the resulting activation output.

It should be noted that while pooling blocks can be used to scale down an image and unpooling blocks can be used to enlarge these scaled down activation maps, convolution and deconvolution blocks can be structured to both convolve/deconvolve and scale down/enlarge without separate pooling and unpooling blocks.

The pooling and unpooling process can be limited depending on the objects of interest being detected in an image input. Since pooling generally scales down an image by looking at sub-image windows without overlap of windows, there is a clear loss in spatial info as the scaling down occurs.

A processing block can include other layers that are packaged with a convolutional or deconvolutional layer. These can include, for example, a rectified linear unit layer (ReLU) or exponential linear unit layer (ELU), which are activation functions that examine the output from a Conv layer in its processing block. The ReLU or ELU layer acts as a gating function to advance only those values corresponding to positive detection of the feature of interest unique to the Conv layer its processing block.

Given a basic architecture, the CNN is then prepared for a training process to hone its accuracy in image classification/detection (of objects of interest). Using training data sets, or sample images used to train the CNN so that it updates its parameters in reaching an optimal, or threshold, accuracy, a process called backpropagation (backprop) occurs. Backpropagation involves a series of repeated steps (training iterations) that, depending on the parameters of the backprop, either will slowly or quickly train the CNN. Backprop steps generally include forward pass, loss function, backward pass, and parameter (weight) update according to a given learning rate. The forward pass involves passing a training image through the CNN. The loss function is a measure of error in the output. The backward pass determines the contributing factors to the loss function. The weight update involves updating the parameters of the filters to move the CNN towards optimal. The learning rate determines the extent of weight update per iteration to arrive at optimal. If the learning rate is too low, the training may take too long and involve too much processing capacity. If the learning rate is too fast, each weight update may be too large to allow for precise achievement of a given optimum or threshold.

The backprop process can cause complications in training, thus leading to the desire for lower learning rates and more specific and carefully determined initial parameters upon start of training. One such complication is that, as weight updates occur at the conclusion of each iteration, the changes to the parameters of the Conv layers amplify the deeper the network goes. For example, if a CNN has a plurality of Conv layers that, as discussed above, allows for higher-level feature analysis, the parameter update to the first Conv layer is multiplied at each subsequent Conv layer. The net effect is that the smallest changes to parameters have large impact depending on the depth of a given CNN. This phenomenon is referred to as internal covariate shift.

It should be noted that even though CNNs are spoken about in detail above, the various embodiments discussed herein could utilize any neural network type or architecture.

In describing the various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various embodiments may have been presented as a group of particular components. However, these embodiments should not be limited to the particular set of elements, nor their specific configuration, communication and physical orientation with respect to each other. One skilled in the art should readily appreciate that these elements can have various configurations and physical orientations (e.g., wholly separate components, units and subunits of groups of components, different communication regimes between components).

Having described the disclosure in detail, it will be apparent that modifications, variations, and equivalent embodiments are possible without departing from the scope of the disclosure defined in the appended claims. Furthermore, it should be appreciated that all examples in the present disclosure are provided as non-limiting examples.

### EXAMPLES

The following non-limiting examples are provided to further illustrate embodiments of the disclosure herein. It should be appreciated by those of skill in the art that the techniques disclosed in the examples that follow represent approaches that have been found to function well in the practice of the disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the disclosure.

### Example 1

### Study Design and Methods

### Derivation and Validation Cohorts

The Genomics of Pediatric Septic Shock cohort in the Sepsis Genomics Collaborative is an ongoing prospective, multicenter observational cohort of critically ill children and has been described extensively.

The institutional review boards of 13 participating hospitals approved the study protocol (Identifier: 2008-0558; initial approval May 9, 2002). Informed consent was obtained from parent or guardian of patients. Patients in the independent test cohort were enrolled at the Children's Hospital of Philadelphia (institutional review board identifiers: 13-010742 [approved Jan. 19, 2014] and 13-010578 [approved Jul. 1, 2014]). All research involving human participants was conducted in accordance with the ethical standards of the institutional review boards and with the tenets of the 1964 Declaration of Helsinki and its later amendments.

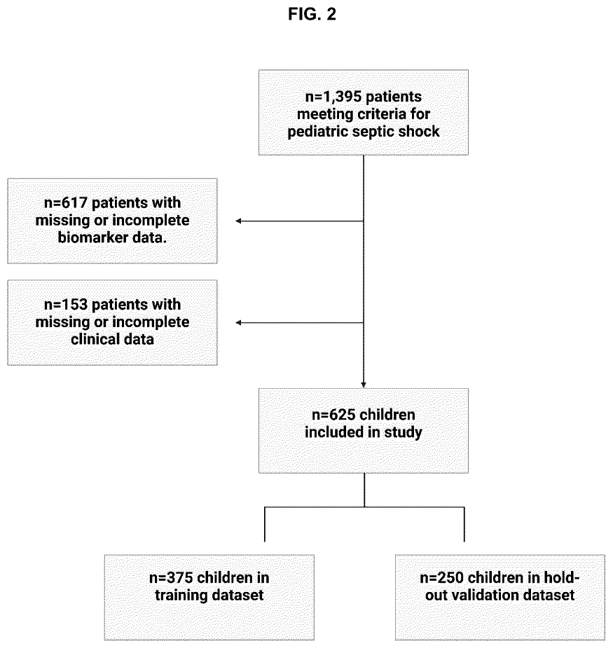

Briefly, patients 0 to 18 years of age meeting consensus criteria for pediatric septic shock were recruited from pediatric intensive care units (PICUs) across the United States between 2003 and 2023. No study-related interventions were undertaken except for blood draws and data collection. Clinical and laboratory data generated during routine care were collected from days 1 through 7, with patient outcomes tracked for 28 days. Notably, this cohort lacked chest radiography data for quantifying lung infiltrates, as well as information on noninvasive respiratory support methods and ventilator data, including mean airway pressure needed to calculate the oxygenation index (OI). Patients with missing clinical and endothelial biomarker data collected within 24 hours of meeting the study criteria were excluded from the analysis, as shown in the study flow diagram shown in FIG. 2.

### Test Cohort

Patients were recruited prospectively through a single-center study of patients with sepsis in the PICU, some of whom met SA ARD criteria. The study was approved by the institution's institutional review board (Identifier: 13-010578) and the study period was from 2014 through 2018. Inclusion criteria included age younger than 18 years, admission to the PICU, sepsis defined according to historical consensus criteria, with or without ARDS as defined by the Berlin criteria. Exclusion criteria included weight <7.5 kg, WBC count <0.5×10³/mL, known mitochondrial disease, baseline hypoxemia, cyanotic heart disease, congestive heart failure, or prolonged mechanical ventilation before enrollment. Patients were enrolled after obtaining informed consent. In this cohort, ARDS definitions were available based on Berlin criteria and details on 01. Blood was collected for plasma biomarker measurement within 24 hours of meeting study criteria and again at a second time point on days 3 through 5. Persistent SA ARD was defined as meeting the SA ARD criteria at the second time point.

### Outcomes

The primary outcome of interest was persistent SA ARD on day 3. This construct was defined as follows. Patients without any preexisting lung disease were classified as having SA ARD if they met 1 of the following criteria: need for endotracheal intubation for acute respiratory failure, mechanical ventilation for >24 hours, PaO₂ to FIO₂ ratio of <250, PaCO₂ of ≥65 mm Hg, or PaO₂ of <40 mm Hg. Patients with a preexisting lung disease were required to meet ≥2 of these criteria to be designated as having SA ARD. The day 3 time point was chosen with the idea that prediction of patients with persistent SA ARD could allow meaningfully for timely interventions in future trials. Secondary outcomes included 28-day mortality; complicated course, that is, a composite outcome of death or ≥2 organ dysfunctions by day 7; and PICU length of stay (LOS) and PICU-free days, calculated by subtracting the PICU LOS from the maximum period of 28 days.

### Endothelial Biomarkers

Endothelial biomarkers measured included angiopoietin 1, angiopoietin 2, tyrosine kinase with immunoglobulin-like loops and epidermal growth factor homology domains 2, soluble thrombomodulin (sTM), IL-8, intercellular adhesion molecule 1, vascular cell adhesion molecule 1 (VCAM-1), and platelet endothelial cell adhesion molecule 1. Serum samples collected on day 1 were analyzed using Luminex assays (R & D Systems) in the derivation cohort. Plasma samples collected on day 1 were measured using a combination of Luminex and ELISA assays in the test cohort.

### Statistical Analyses

Minitab version 21.1.0 software and GraphPad Prism version 9 software were used for data analysis and figure generation. Demographic data were summarized using numbers and percentages or medians with interquartile ranges.

Differences between groups were assessed using the c2 test for categorical variables and the nonparametric Kruskal-Wallis test for continuous variables. Univariable associations between predictor variables and outcomes were tested using binary logistic regression. Multivariable regression models then were constructed with 6 clinical variables: (1) patient age, (2) Pediatric Risk of Mortality III score, (3) presence of comorbidity, (4) immunocompromised status, (5) PaO₂ to FIO₂ ratio of <250, and (6) presence of SA ARD on day 1. For this study, preexisting comorbid medical conditions were categorized by the organ system affected (e.g., heart, lung, neurologic, kidney, gastrointestinal or liver, endocrine, hematologic, cancer, bone marrow, and solid organ transplantation). This was coded as a binary variable indicating the presence (yes) or absence (no) of comorbidity.

Candidate endothelial biomarkers were selected based on independent association with the primary outcome using backward elimination with an a value of 0.1. A 2-tailed P value of <0.05 was considered statistically significant.

### Risk Prediction Modeling

Patients in the derivation cohort were split randomly in a 60:40 ratio into training and holdout validation datasets. This approach was chosen, rather than splitting based on year of enrollment or study site, to ensure that the models were exposed to a representative mix of data, improving their ability to generalize to new and unseen data.

TreeNet, a stochastic gradient boosting method, and Classification and Regression Tree (CART) were used to develop predictive models for day 3 SA ARD (using clinical variables and select endothelial biomarkers. TreeNet builds multiple CART trees iteratively, using residuals from previous trees to enhance predictive accuracy and to capture subtle patterns. However, TreeNet models are less interpretable because contributions are distributed across many iterations and the black box nature of the algorithms used. In contrast, CART provides clear decision rules, revealing how specific combinations of predictors influence outcomes. Thus, both methods are complementary: TreeNet enhances accuracy and generalization by capturing complex patterns, whereas CART aids in identifying key interactions and provides actionable decision rules.

For TreeNet models, class weights were not applied, and interactions of all orders were allowed. A total of 300 trees were grown, with a minimum of 3 and a maximum of 6 cases per terminal node. The optimal number of trees was selected based on minimum misclassification rate. To mitigate overfitting, a learning rate of 0.01 was used. Class weights were also not used for CART models. Node splitting was based on the class probability method, and the optimal tree within k=1 SEs of the minimum misclassification cost was selected. A minimum of 15 cases per terminal node was required for CART models.

### Validation

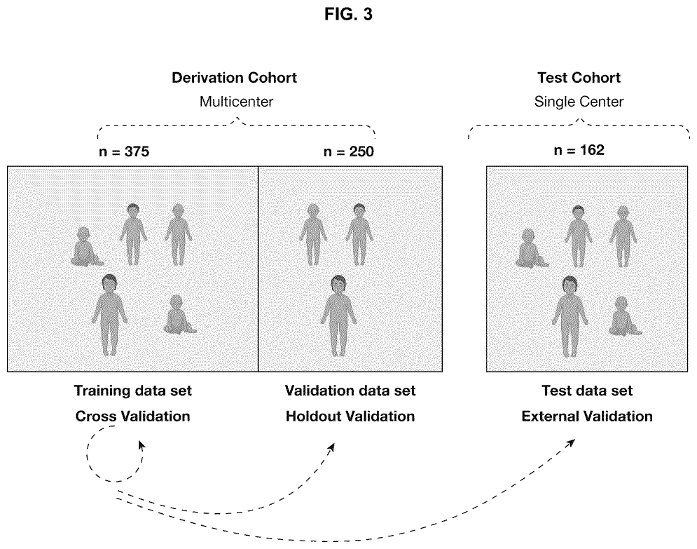

Because of congruence in outputs between TreeNet and CART models in the derivation cohort, we selected the CART model for validation, prioritizing its simplicity, interpretability, and practicality for clinical application. A 10-fold cross-validation approach was used within the training dataset. Only the CART model was tested in the holdout validation dataset of the derivation cohort and in the independent test cohort, stratifying patients as high-risk or low-risk for day 3 SA ARD. Clinical outcomes were compared across patient risk strata to assess prognostic usefulness and reproducibility of the model. The overall study structure and scheme for validation are illustrated in FIG. 3.

### Model Performance Metrics

Model performance was evaluated using the area under the receiver operating characteristic curve (AUC), which measures the model's ability to discriminate between high-risk and low-risk patients, as well as diagnostic test characteristics. Model accuracy and reliability was determined by Brier scores, which were calculated with the R package scoring. Brier scores range from 0 to 1, with lower scores indicating better calibration, which means that predicted probabilities aligned closely with observed outcomes.

### Pre-Existing Lung Disease

Patients with a history of any of the 10 following co-morbid conditions were categorized as having pre-existing lung disease: (1) Severe Neurologic Injury with Tracheostomy and Ventilator Dependence, (2) Chronic Respiratory Failure, (3) Chronic Lung Disease (CLD), (4) Bronchopulmonary Dysplasia (BPD), (5) Restrictive lung disease, (6) Pulmonary hypertension, (7) Alveolar hypoventilation syndrome, (8) Cystic fibrosis, (9) Bronchiolitis obliterans and (10) Pulmonary hemosiderosis.

### Choice of Biomarkers

A combination of previously measured biomarkers was chosen in the derivation and test cohorts. This included evaluation of Interleukin-8, a key pro-inflammatory chemokine that recruits neutrophils to sites of infection or injury, which is a critical component of the acute inflammatory response. IL-8 is particularly relevant in the context of lung injury and sepsis, where neutrophil-driven inflammation contributes to endothelial damage and increased vascular permeability. This additionally included evaluation of Angpt-1, Angpt-2, and their common receptor Tie2, which together regulate the stability and permeability of the vascular endothelium. Angpt-2 counteracts A ngpt-1 and destabilizes the endothelium, and increased concentrations of Angpt-2 are associated with increased odds of mortality in multiple pediatric cohorts. Several adhesion molecules, including ICAM-1, VCAM-1, and PECAM-1, that are involved in immune-cell migration to the site of injury, were also included. ICAM-1 has been associated with the severity of lung injury, length of mechanical ventilation, and mortality, while VCAM-1 has not been well studied in the pediatric population, but is associated with ventilator-induced lung injury in adults. Finally, the analysis included soluble thrombomodulin, an anti-thrombotic protein expressed on the surface of the endothelium that is cleaved in response to endothelial injury and has been associated with increased mortality in pediatric cohorts.

### Example 2

### Clinical Characteristics of Patients With and Without Persistent SA ARD

The demographic features, clinical characteristics, and outcomes of interest in patients with (n=219) and without (n=156) day 3 SA ARD in the training dataset are presented in Table 1. Patients with day 3 SA ARD were younger, less commonly were female, and had a higher illness severity at disease onset. Patients with day 3 SA ARD experienced worse clinical outcomes, including 28-day mortality, complicated course, longer PICU LOS, and fewer PICU-free days compared with those without day 3 SA ARD. Those with day 3 SA ARD also were more likely to remain intubated, to require mechanical ventilation, and to require continuous renal replacement therapy on day 7 of illness compared with those without day 3 SA ARD. Patients with day 3 SA ARD were more likely to have a pulmonary source of infection compared with those without.

TABLE 1
Demographic data and clinical characteristics
in the derivation set among patients with and
without D 3 SA-ARDS in the training dataset.

| | D 3 SA-ARD | | No D 3 SA-ARD | | |
|---|---|---|---|---|---|
| Variable | (n = 219) | | (n = 156) | | P value |
| Age (Years) | 3.1 | (0.8, 7.4) | 6.5 | (3.3, 13.0) | <0.001 |
| Sex, Female (%) | 95 | (43.4%) | 86 | (55.2%) | 0.025 |
| Race (Self-identified) | | | | | 0.37 |
| White/Caucasian | 157 | (71.7%) | 117 | (75.0%) | |
| Black/African | 30 | (13.7%) | 14 | (9.0%) | |
| American | | | | | |
| Other | 32 | (14.6%) | 25 | (16.0%) | |
| Ethnicity | | | | | 0.425 |
| Hispanic or Latino | 33 | (15.1%) | 19 | (12.2%) | |
| PRISM-III | 12 | (6, 19) | 9 | (5, 14) | 0.003 |
| 28-day Mortality | 43 | (19.6%) | 4 | (2.6%) | <0.001 |
| Complicated course | 121 | (55.3%) | 9 | (5.8%) | <0.001 |
| PICU LOS | 8 | (5, 15) | 3 | (1, 7) | <0.001 |
| PICU Free Days | 20 | (13, 23) | 25 | (21, 27) | <0.001 |
| Day 7 MV | 113 | (71.5%) | 8 | (17.8%) | <0.001 |
| Day 7 Intubated | 61 | (38.6%) | 0 | (0) | <0.001 |
| Day 7 P/F <250 | 33 | (20.9%) | 0 | (0) | <0.001 |
| Day 7 CRRT* | 24 | (11.0%) | 3 | (1.9%) | <0.001 |
| Day 7% PFO Day 1-7 | 6.9% | (−0.5, 15.2) | 3.6% | (0, 8.6) | 0.023 |
| Day 1-7 Cardiac | 43 | (19.6%) | 4 | (2.6%) | <0.001 |
| Arrest | | | | | |
| Positive blood | 37 | (16.9%) | 31 | (19.9%) | 0.461 |
| culture | | | | | |
| Positive culture | 137 | (62.6%) | 80 | (51.3%) | 0.029 |
| Source | | | | | <0.001 |
| Pulmonary | 69 | (31.5%) | 17 | (10.9%) | |
| Extra-pulmonary | 68 | (31.1%) | 63 | (40.4%) | |
| Steroids | 124 | (56.6%) | 72 | (46.2%) | 0.045 |

*Adjusted for mortality prior to day 7.

### Example 3

### Association Between Predictor Variables and Persistent SA ARD

Clinical variables for inclusion in the risk prediction models included the patient age, PRISM-III severity of illness score, PaO₂/FIO₂ ratio, and the presence of SA-ARD on day 1. Concentrations of Angiopoietin-1 (Angpt-1), Angiopoietin-2 (Angpt-2), Tyrosine kinase with immunoglobulin-like loops and Epidermal growth factor homology domains-2 (Tie-2), soluble Thrombomodulin (sTM), Interleukin-8 (IL-8), Inter-Cellular Adhesion Molecule-1 (ICA M-1), Vascular Cell Adhesion Molecule-1 (VCAM-1), and Platelet Endothelial Cell Adhesion Molecule (PECAM-1) in pg/mL were previously measured in serum collected on day 1 of septic shock by Luminex assays (R & D Systems, MN).

Univariable and multivariable associations between predictor variables and day 3 SA ARD in the training dataset are detailed in Table 2 and Table 3. Age, Pediatric Risk of Mortality III scores, presence of comorbidity, immunocompromised status, PaO 2 to FIO₂ ratio of <250, and presence of SA ARD on day 1 all were associated with day 3 SA ARD. Increments in angiopoietin 2, angiopoietin 2 to angiopoietin 1 ratio, angiopoietin 2 to tyrosine kinase with immunoglobulin-like loops and epidermal growth factor homology domains 2 ratio, IL-8, sTM, platelet endothelial cell adhesion molecule 1, and intercellular adhesion molecule 1 were associated with increased odds of day 3 SA ARD, whereas increments in VCAM-1 were associated with a decreased odds of day 3 SA ARD. In multivariable models with adjustment of clinical variables in the models, sTM, VCAM-1, and angiopoietin 1 remained independently associated with outcome of interest.

TABLE 2
Univariate associations between clinical and endothelial
biomarker variables tested and presence of sepsis
associated acute respiratory dysfunction on day 3
(D 3 SA-ARD) in the training dataset (n = 375).

| | Odds ratio | P value |
|---|---|---|
| Clinical variables | | |
| Age | 0.92 (0.89, 0.95) | <0.001 |
| PRISM-III | 1.05 (1.02) | <0.001 |
| Pre-existing Comorbidity | 0.52 (0.33, 0.77) | 0.002 |
| Immunocompromised | 0.49 (0.29, 0.83) | 0.008 |
| P/F Ratio <250 | 16.1 (6.3, 40.8) | <0.001 |
| D 1 SA-ARD | 32.0 (16.8, 60.5) | <0.001 |
| Endothelial biomarkers | | |
| Angpt-1 (log10) | 0.89 (0.57, 1.39) | 0.606 |
| Angpt-2 (log10) | 2.75 (1.65, 4.58) | <0.001 |
| Tie-2 (log10) | 0.99 (0.47, 2.06) | 0.973 |
| Angpt-2/Angpt-1 ratio | 1.07 (1.01, 1.13) | 0.017 |
| Angpt-2/Tie-2 ratio | 1.18 (1.02, 1.36) | 0.021 |
| IL-8 (log10) | 1.74 (1.32, 2.29) | <0.001 |
| Soluble thrombomodulin (log10) | 4.41 (1.80, 10.8) | 0.001 |
| PECAM-1 (log10) | 3.42 (1.37, 8.54) | 0.009 |
| ICAM-1 (log10) | 2.93 (1.18, 7.25) | 0.020 |
| VCAM-1 (log10) | 0.40 (0.18, 0.88) | 0.022 |
| E-Selectin (log10) | 0.69 (0.32, 1.52) | 0.364 |
| P-Selectin (log10) | 1.54 (0.54, 4.38) | 0.420 |

TABLE 3
Biomarkers selected based on multivariate associations
with backward elimination between and presence of sepsis
associated acute respiratory dysfunction on day 3 (D
3 SA-ARD) in the training dataset (n = 375).

| Endothelial biomarkers | Odds ratio | P value |
|---|---|---|
| Angpt-1 (log10) | 0.41 (0.20, 0.84) | 0.016 |
| VCAM-1 (log10) | 0.18 (0.05, 0.61) | 0.005 |
| sTM (log10) | 5.98 (1.5, 23.7) | 0.011 |

All clinical variables were coerced into the model. Alpha of 0.1 was used for backward elimination of biomarkers.

### Example 4

### Derivation of Risk Prediction Models Including Clinical Variables and Endothelial Biomarkers for Persistent SA ARD

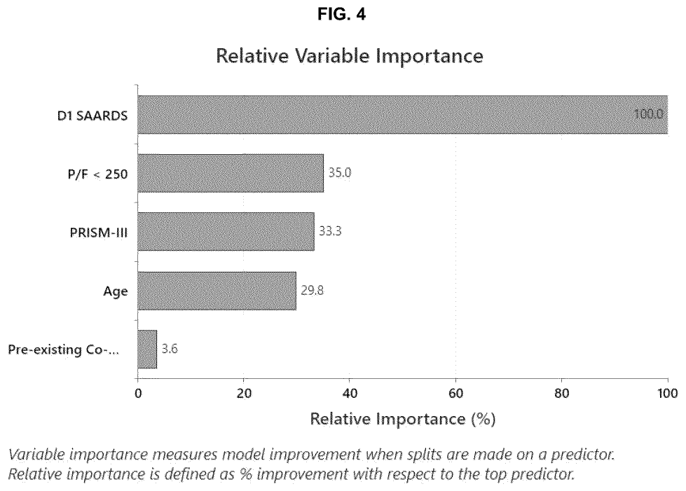

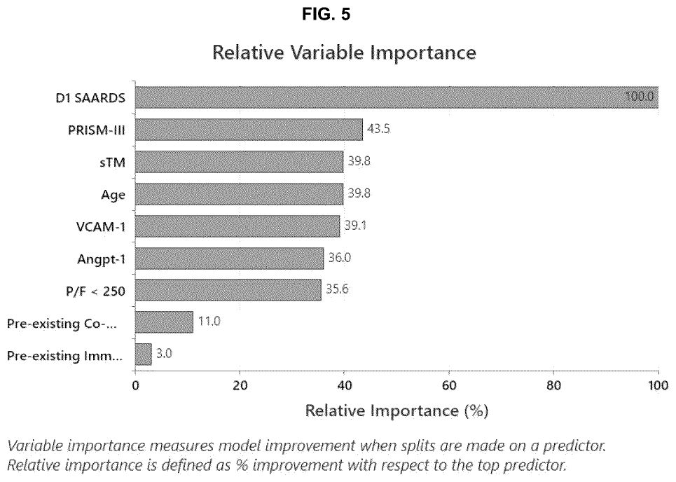

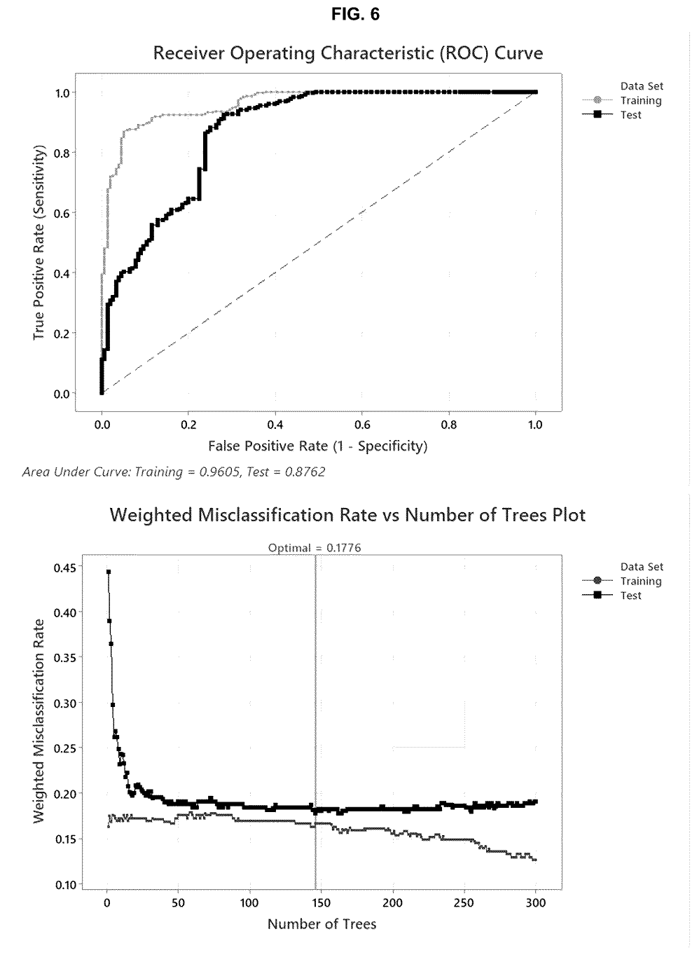

Model summary and confusion matrix of a TreeNet model including only clinical variables predictive of day 3 SA ARD in the training dataset are detailed in Tables 4 and 5, with relative variable importance shown in FIGS. 4 and 5. The TreeNet model incorporating endothelial biomarkers predictive of day 3 SA ARD is detailed in Tables 6 and 7 and FIGS. 4 and 5. This model had an AUC of 0.96 (95% CI, 0.95-0.98) and 0.87 (95% CI, 0.83-0.90) on 10-fold cross-validation (FIG. 6).

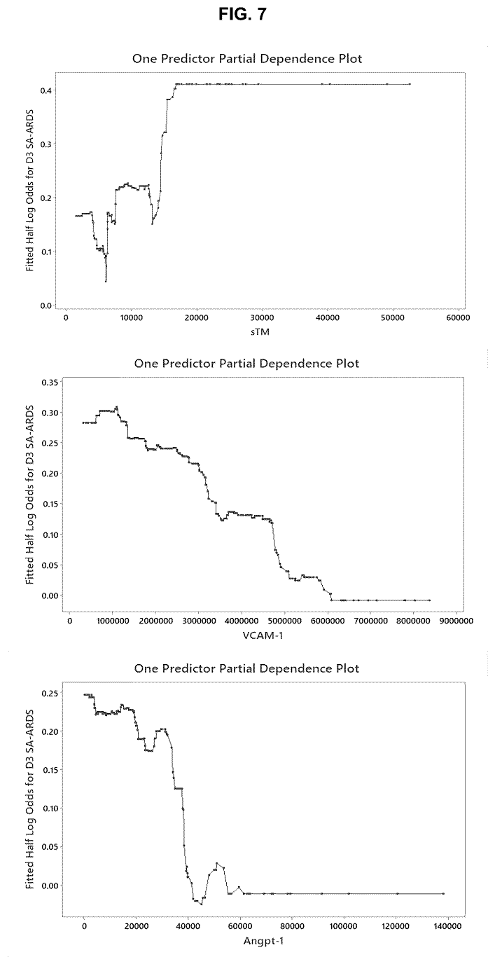

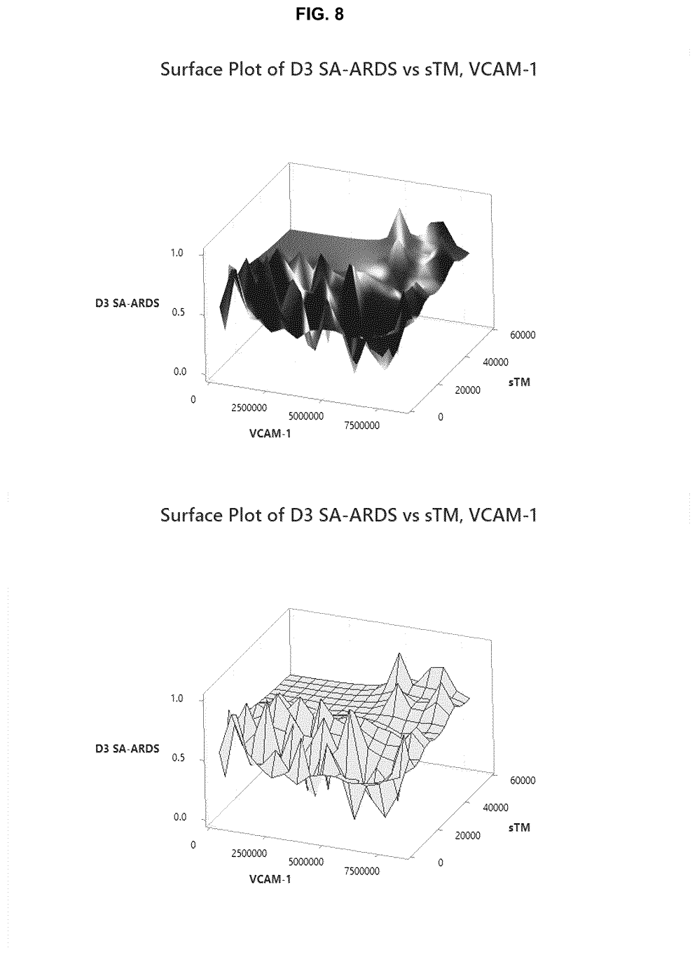

Although the model inclusive of biomarkers showed comparable sensitivity (93.15% vs 93.61%) and specificity (73.7% vs 69.2%) as models without biomarkers, the misclassification and false-positive rates were lower with inclusion of the biomarkers. The biomarkers in a decreasing order of relative importance were sTM, VCAM-1, and angiopoietin 1, with respective one-predictor partial dependence plots shown in FIG. 7. Notably, patients with low sTM and high VCAM-1 concentrations were identified as having a lower Q13 odds of day 3 sepsis-associated ARDS in the training dataset, as illustrated in the surface plot in FIG. 8.

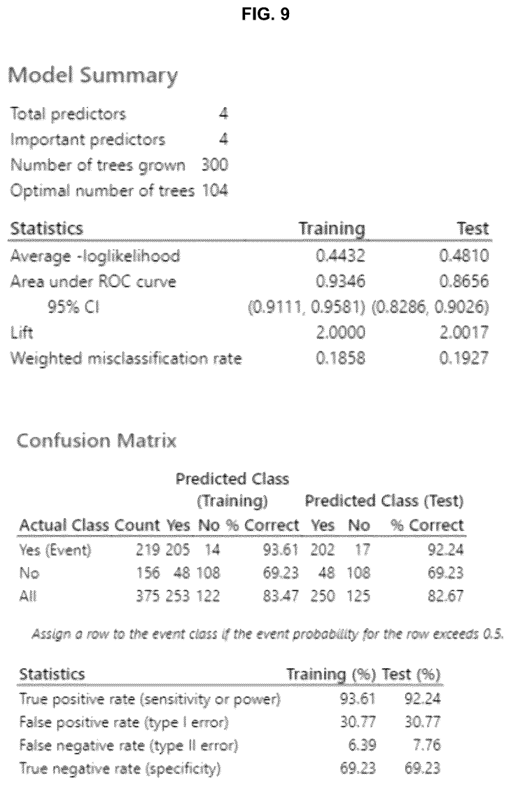

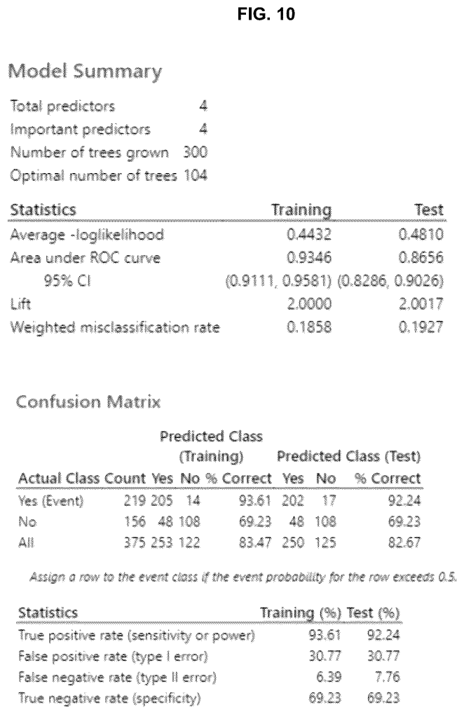

The TreeNet® model predictive of D3 SA-ARD in the derivation dataset is detailed in FIGS. 4-6, 9, and 10. The top predictor variables chosen by the model were the presence of D1 SA-ARD and P/F ratio <250. The biomarkers in order of decreasing relative variable importance included sTM, A ngpt-2/A ngpt-1, VCAM-1, and IL-8. TreeNet model identified an interaction between sTM and VCAM-1 concentrations on log odds of D3 SA-ARD. Patients with high sTM and VCAM-1 concentrations had increased odds of D3 SA-ARD. However, a subset of patients with low sTM concentrations and high VCAM-1 concentrations had a lower odds of D3 SA-ARD, as shown in the surface plot in FIG. 8 of the Online Supplement. The TreeNet® model had an AUROC of 0.96 (95% CI: 0.95-0.98) and 0.87 (95% CI: 0.830.90) upon 10-fold cross-validation as shown in FIG. 6. The model had a sensitivity of 82.7% and specificity of 93.7%.

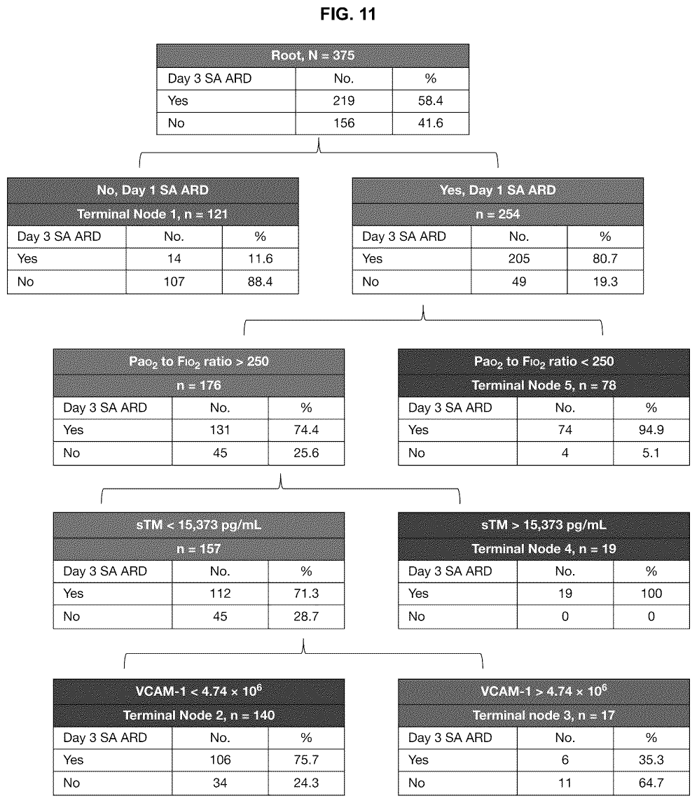

FIG. 11 shows the CART model predictive of day 3 SA ARD in the training dataset. Consistent with the TreeNet model, the presence of day 1 SA ARD and PaO₂ to FIO₂ ratio of <250 were key decision points at the top of the tree. Patients who did not have day 1 SA ARD were unlikely to have day 3 SA ARD (terminal node 1, 11.6%). Patients with day 1 SA ARD and PaO₂ to FIO₂ ratio of <250 had a high rate of day 3 SA ARD (terminal node 5, 94.9%). Among patients with day 1 SA ARD but a PaO₂ to FIO₂ ratio of >250, all patients with sTM concentrations of ≤15,373 pg/mL had day 3 SA ARD (terminal node 4, 100%). Among patients with sTM concentrations of ≤15,374 pg/mL, patients with VCAM-1 concentration <4.7×10⁶ pg/mL had a higher rate of day 3 SA ARD (terminal node 2, 75.7%). In contrast, those with VCAM-1 concentration of >4.7×10⁶ pg/mL had a relatively low rate of day 3 SA ARD (terminal node 3, 35.3%).

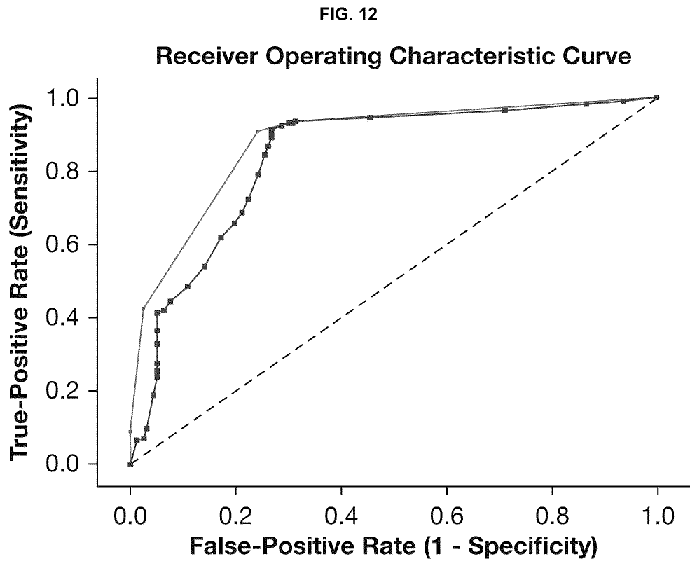

The AUC of the CART model inclusive of endothelial biomarkers was 0.88 and was 0.83 on 10-fold cross-validation in the training dataset (FIG. 12). The model showed comparable sensitivity of 90.8% (95% CI, 86.1%-94.2%) vs 93.6% (95% CI, 89.2%-96.3%) and higher specificity of 75.6% (95% CI, 68.0%-81.2%) vs 68.5% (95% CI, 60.6%-75.6%) as compared with CART models without endothelial biomarkers (data not shown). Brier scores for the TreeNet and CART models in the training dataset were 0.147 and 0.154, respectively.

TABLE 4
Model summary of TreeNet ® Model inclusive of only
clinical variables including (1) age, (2) PRISM-III score,
(3) pre-existing comorbidity, (4) history of immunocompromised status,
(5) presence of sepsis associated acute respiratory dysfunction on day 1
(D 1 SA-ARD), and (6) PaO₂ to FIO₂ ratio <250 to predict
risk of day 3 sepsis associated acute respiratory dysfunction
(D 3 SA-ARD) in the training dataset (n = 375).

| | |
|---|---|
| Total predictors | 6 |
| Important predictors | 5 |
| Number of trees grown | 300 |
| Optimal number of trees | 37 |

| Statistics | Training | Test |
|---|---|---|
| Average -loglikelihood | 0.5594 | 0.5735 |
| Area under ROC curve | 0.9151 | 0.8766 |
| 95% CI | (0.8868, 0.9434) | (0.8389, 0.9143) |
| Lift | 2 | 1.9364 |
| Weighted | 0.1858 | 0.1872 |
| misclassification rate | | |

TABLE 5
Confusion Matrix of TreeNet ® Model inclusive of only
clinical variables including (1) age, (2) PRISM-III score,
(3) pre-existing comorbidity, (4) history of immunocompromised status,
(5) presence of sepsis associated acute respiratory dysfunction on day 1
(D 1 SA-ARD), and (6) PaO₂ to FIO₂ ratio <250 to predict
risk of day 3 sepsis associated acute respiratory dysfunction
(D 3 SA-ARD) in the training dataset (n = 375).

| | | Predicted Class | | | Predicted Class | | |
|---|---|---|---|---|---|---|---|
| | | (Training) | | | (Test) | | |
| Actual | | | | % | | | % |
| Class | Count | 1 | 0 | Correct | 1 | 0 | Correct |
| 1.00 | 219 | 205 | 14 | 93.61 | 203 | 16 | 92.69 |
| (Event) | | | | | | | |
| 0 | 156 | 48 | 108 | 69.23 | 47 | 109 | 69.87 |
| All | 375 | 253 | 122 | 83.47 | 250 | 125 | 83.2 |

| Statistics | Training (%) | Test (%) |
|---|---|---|
| True positive rate (sensitivity or power) | 93.61 | 92.69 |
| False positive rate (type I error) | 30.77 | 30.13 |
| False negative rate (type II error) | 6.39 | 7.31 |
| True negative rate (specificity) | 69.23 | 69.87 |

TABLE 6
Model summary of TreeNet ® Model inclusive of
all clinical variables and selected endothelial biomarkers
with independent association with outcome of interest
to predictive of day 3 sepsis associated acute respiratory
dysfunction (D 3 SA-ARD) in the training dataset (n = 375).

| | |
|---|---|
| Total predictors | 9 |
| Important predictors | 8 |
| Number of trees grown | 300 |
| Optimal number of trees | 146 |

| Statistics | Training | Test |
|---|---|---|
| Average -loglikelihood | 0.3897 | 0.4573 |
| Area under ROC curve | 0.9605 | 0.8762 |
| 95% CI | (0.9434, 0.9776) | (0.8384, 0.9139) |
| Lift | 2 | 2.0017 |
| Weighted | 0.1657 | 0.1776 |
| misclassification rate | | |

TABLE 7
Confusion Matrix of TreeNet ® Model inclusive of
all clinical variables and selected endothelial biomarkers
with independent association with outcome of interest to
predictive of day 3 sepsis associated acute respiratory
dysfunction (D 3 SA-ARD) in the training dataset (n = 375).

| | | Predicted Class | | | Predicted Class | | |
|---|---|---|---|---|---|---|---|
| | | (Training) | | | (Test) | | |
| Actual | | | | % | | | % |
| Class | Count | 1 | 0 | Correct | 1 | 0 | Correct |
| 1.00 | 219 | 204 | 15 | 93.15 | 203 | 16 | 92.69 |
| (Event) | | | | | | | |
| 0 | 156 | 41 | 115 | 73.72 | 44 | 112 | 71.79 |
| All | 375 | 245 | 130 | 85.07 | 247 | 128 | 84 |

| Statistics | Training (%) | Test (%) |
|---|---|---|
| True positive rate (sensitivity or power) | 93.15 | 92.69 |
| False positive rate (type I error) | 26.28 | 28.21 |
| False negative rate (type II error) | 6.85 | 7.31 |
| True negative rate (specificity) | 73.72 | 71.79 |

### Example 5

### Validation and Testing of Risk Prediction Models Including Clinical Variables and Endothelial Biomarkers for Persistent SA ARD

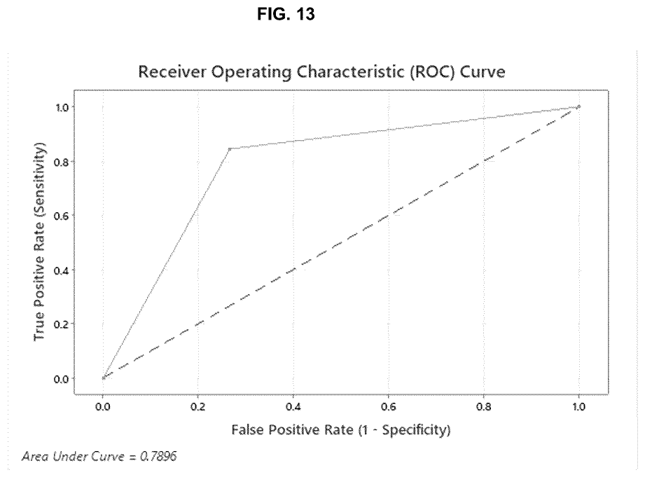

The performance of the CART model for day 3 SA ARD was assessed among 250 patients in the holdout validation set, as shown in Table 8. The AUC of the model to predict day 3 SA ARD was 0.79 (FIG. 13), with a positive predictive value of 84% and a negative predictive value of 74%. Model Brier score in this dataset was 0.197.

One hundred fifty-six patients (62.7%) were designated as high risk based on assignment to terminal nodes 2, 4, and 5, and 93 patients (37.3%) were designated as low risk for day 3 SA ARD based on assignment to terminal nodes 1 and 3. Patients deemed high risk for day 3 SA ARD were more likely to have complicated course, longer PICU LOS, and fewer PICU-free days. One hundred thirty-one patients (85.1%) classified as high risk actually had day 3 SA ARD, compared with 21 patients (30.0%) in the low-risk group (P<0.001). No differences in mortality, rate of sepsis-associated acute kidney injury, or need for continuous renal replacement therapy were found between risk strata.

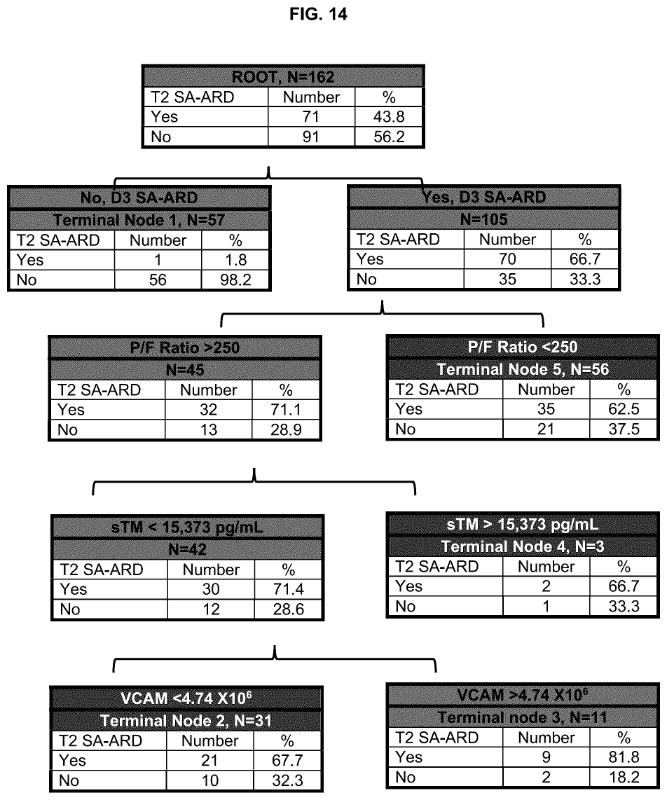

The test cohort comprised 162 pediatric patients admitted to the PICU with sepsis. The demographic and clinical characteristics of this cohort based on the presence of persistent SA ARD are presented in Table 9. The rate of persistent day 3 SA ARD by terminal node of the CART model in the test cohort is shown in FIG. 14. As shown in the figure and in Table 10, among 90 patients classified as high risk based on the CART model (terminal nodes 2, 4, and 5), 58 patients (64.5%) had day 3 SA ARD in the test cohort relative to 10 of 68 patients (14.7%) among those classified as low risk (terminal nodes 1 and 3).

Table 11 shows outcomes of patients classified as high risk and low risk of persistent SA ARD in the test cohort. Notably, those classified as being at high risk of persistent SA ARD showed a greater need for mechanical ventilation on day 3 (71.1% vs 38.9%; P<0.001) and day 7 (43.3% vs 15.2%; P ¼. 007) and included a higher proportion of patients with a PaO₂ to FIO₂ ratio of <250 at the latter time point (17% vs 0%; P ¼ 0.048). Although the OI was numerically higher among patients classified as being at high risk, no statistically significant differences were found between the groups at either day 3 or 7 in the test cohort.

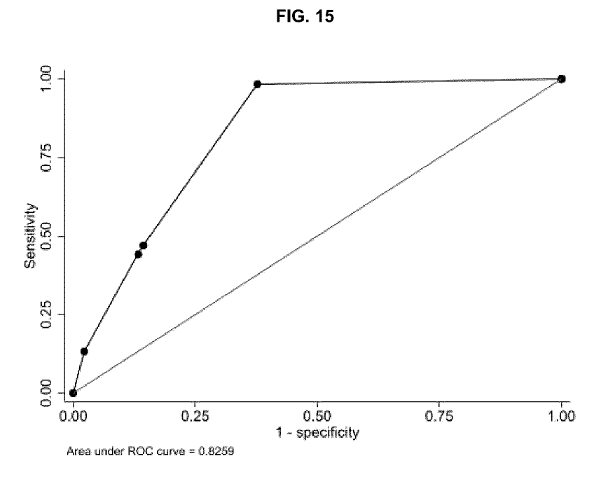

The AUC of the CART model in the test cohort was 0.83 (FIG. 15). The CART model had a Brier score of 0.14 in the test cohort. The diagnostic test characteristics of the CART model inclusive of clinical variables and endothelial biomarkers in the training, validation, and test datasets are summarized in Table 12.

TABLE 8
Clinical characteristics of patients classified as high- and
low-risk of day 3 sepsis associated acute respiratory dysfunction
(D 3 SA-ARD) in the hold-out validation dataset (n = 250).

| | High D 3 SA-ARD | | Low D 3 SA-ARD | | |
|---|---|---|---|---|---|
| Variable | risk (n = 156) | | risk (n = 93) | | p value |
| Age (Years) | 3.5 | (1.1, 7.9) | 7.3 | (2.9, 12.1) | <0.001 |
| Sex, Female (%) | 73 | (46.8) | 47 | (50.4%) | 0.567 |
| Race (Self- | | | | | |
| identified) | | | | | |
| White/Caucasian | 117 | (75.0%) | 67 | (72.0%) | 0.867 |
| Black/African | 20 | (12.8%) | 10 | (10.7%) | |
| American | | | | | |
| Other | 16 | (12.2%) | 16 | (17.2%) | |
| Ethnicity | | | | | |
| Hispanic or Latino | 4 | (2.6%) | 0 | (0) | |
| PRISM-III | 11 | (7, 16) | 10 | (5, 13) | 0.104 |
| 28-day Mortality | 8 | (5.3%) | 9 | (9.6%) | 0.176 |
| Complicated course | 54 | (34.6%) | 16 | (17.1%) | 0.003 |
| PICU LOS | 8 | (5, 16) | 2 | (1, 9) | <0.001 |
| PICU Free Days | 20 | (11, 23) | 26 | (19, 27) | <0.001 |
| D 3 SA-ARD | 131 | (85.1%) | 21 | (30.0%) | <0.001 |
| D 7 SA-ARD | 68 | (61.8%) | 11 | (32.3%) | 0.003 |
| D 3 MV | 129 | (83.8%) | 27 | (39.2%) | <0.001 |
| D 7 MV | 71 | (64.5%) | 16 | (47.1%) | 0.068 |
| D 3 P/F <250 | 35 | (22.8%) | 4 | (5.8%) | 0.002 |
| D 7 P/F <250 | 21 | (19.1%) | 1 | (3.0%) | 0.022 |
| D 3 SA-AKI | 51 | (33.2%) | 21 | (30.0%) | 0.643 |
| D 7 SA-AKI | 38 | (34.6%) | 13 | (38.2%) | 0.694 |
| Any CRRT | 17 | (10.9%) | 10 | (10.8%) | 0.972 |
| D 3 CRRT | 15 | (9.6%) | 6 | (6.5%) | 0.385 |
| D 7 CRRT | 12 | (7.7%) | 8 | (8.6%) | 0.798 |

TABLE 9
Comparison of patients with and without sepsis associated
acute respiratory dysfunction at timepoint 2 (T2
SA-ARD) in the test cohort (n = 162).

| | T2 SA-ARD | | No T2 SA-ARD | | |
|---|---|---|---|---|---|
| Variable | (n = 71) | | (n = 91) | | P value |
| Age (Years) | 9.9 | (2.9, 14.1) | 6.8 | (3.3, 14.5) | 0.552 |
| Sex, Female (%) | 31 | (43.7%) | 49 | (53.9%) | 0.198 |
| Race (Self- | | | | | 0.602 |
| identified) | | | | | |
| White/Caucasian | 35 | (49.3%) | 46 | (50.6%) | |
| Black/African | 16 | (22.5%) | 25 | (27.5%) | |
| American | | | | | |
| Other | 20 | (28.2%) | 20 | (22.0%) | |
| Ethnicity | | | | | 0.865 |
| Hispanic or Latino | 11 | (15.5%) | 11 | (16.5%) | |
| PRISM-III | 14 | (7, 19) | 10 | (5, 15) | 0.013 |
| PICU Mortality | 11 | (15.5%) | 8 | (8.8%) | 0.188 |
| Complicated course | 67 | (94.4%) | 46 | (50.6%) | <0.001 |
| PICU LOS | 15 | (9, 28) | 7 | (3, 16) | <0.001 |
| PICU Free Days | 13 | (0, 19) | 21 | (12, 25) | <0.001 |
| Day 7 MV | 71 | (100%) | 24 | (42.1) | <0.001 |
| Day 7 Intubation | 71 | (100%) | 24 | (42.1) | <0.001 |
| Day 7 P/F <250 | 28 | (43.1%) | 1 | (5.9) | 0.004 |
| Day 7 CRRT* | 2 | (20%) | 0 | (0%) | 0.092 |
| Day 1-7% PFO | 11.5% | (5.7, 18.5) | 8.5% | (3.4, 20.0) | 0.179 |
| Day 1-7 Cardiac | N/A | | N/A | | |
| Arrest | | | | | |
| Positive blood | 15 | (21.1%) | 28 | (30.8%) | 0.168 |
| culture | | | | | |
| Positive culture | 51 | (71.8%) | 63 | (69.2%) | 0.719 |
| Source | | | | | 0.255 |
| Pulmonary | 24 | (47.1%) | 23 | (36.5%) | |
| Extra-pulmonary | 27 | (52.9%) | 40 | (63.5%) | |
| Steroids | 37 | (57.8%) | 39 | (70.9%) | 0.138 |

TABLE 10
Clinical characteristics of patients classified as high- and
low-risk of persistent sepsis associated acute respiratory
dysfunction (T2 SA-ARD) in the test dataset (n = 158).

| | High D 3 SA-ARD | | Low D 3 SA-ARD | | |
|---|---|---|---|---|---|
| Variable | risk (n = 90) | | risk (n = 68) | | P value |
| D 3 MV | 64/73 | (87.7%) | 28/51 | (54.9%) | <0.001 |
| D 3 P/F <250 | 25/64 | (39.1%) | 4/17 | (23.5%) | 0.20 |
| D 3 OI | 6.1 (3.7, 9.5) | | 4.8 (3.2, 7.0) | | 0.40 |
| | for n = 55 | | for n = 10 | | |
| D 7 MV | 39/60 | (65.0%) | 11/31 | (35.5%) | 0.007 |
| D 7 Non- | 3/26 | (11.5%) | 0/4 | (0%) | 0.2 |
| Conventional | | | | | |
| Ventilator Mode | | | | | |
| D 7 P/F <250 | 17/24 | (70.8%) | 0/6 | (0%) | 0.048 |

| | | | |
|---|---|---|---|
| D 7 OI | 5.7 (3.7, 8.4) | 5.0 (2.9, 5.2) | 0.2 |
| | for n = 3 | for n = 33 | |

TABLE 11
Clinical characteristics of patients classified as high- and
low-risk of day 3 sepsis associated acute respiratory dysfunction
(D 3 SA-ARD) in the validation dataset (n = 249).

| | High D 3 SA-ARD | | Low D 3 SA-ARD | | |
|---|---|---|---|---|---|
| Variable | Risk (n = 156) | | Risk (n = 93) | | P value |
| Age (Years) | 3.5 | (1.1, 7.9) | 7.3 | (2.9, 12.1) | <0.001 |
| Sex, Female (%) | 73 | (46.8) | 47 | (50.4%) | 0.567 |
| Race (Self- | | | | | 0.37 |
| identified) | | | | | |
| White/Caucasian | 117 | (75.0%) | 67 | (72.0%) | 0.867 |
| Black/African | 20 | (12.8%) | 10 | (10.7%) | |
| American | | | | | |
| Other | 16 | (12.2%) | 16 | (17.2%) | |
| Ethnicity | | | | | 0.425 |
| Hispanic or Latino | 4 | (2.6%) | 0 | (0) | |
| PRISM-III Score | 11 | (7, 16) | 10 | (5, 13) | 0.104 |
| 28-day Mortality | 8 | (5.3%) | 9 | (9.6%) | 0.176 |
| Complicated course | 54 | (34.6%) | 16 | (17.1%) | 0.003 |
| PICU LOS | 8 | (5, 16) | 2 | (1, 9) | <0.001 |
| PICU Free Days | 20 | (11, 23) | 26 | (19, 27) | <0.001 |
| D 3 SA-ARD | 131 | (85.1%) | 21 | (30.0%) | <0.001 |
| D 7 SA-ARD | 68 | (61.8%) | 11 | (32.3%) | 0.003 |
| D 3 MV | 129 | (83.8%) | 27 | (39.2%) | <0.001 |
| D 7 MV | 71 | (64.5%) | 16 | (47.1%) | 0.068 |
| D 3 P/F <250 | 35 | (22.8%) | 4 | (5.8%) | 0.002 |
| D 7 P/F <250 | 21 | (19.1%) | 1 | (3.0%) | 0.022 |
| D 3 SA-AKI | 51 | (33.2%) | 21 | (30.0%) | 0.643 |
| D 7 SA-AKI | 38 | (34.6%) | 13 | (38.2%) | 0.694 |
| Any RRT | 17 | (10.9%) | 10 | (10.8%) | 0.972 |
| D 3 CRRT | 15 | (9.6%) | 6 | (6.5%) | 0.385 |
| D 7 CRRT | 12 | (7.7%) | 8 | (8.6%) | 0.798 |

Data are presented as No. (%) or median (interquartile range) unless otherwise indicated.
PRISM III: Pediatric risk of mortality score III
LOS: Length of stay
SA-ARD: Sepsis associated acute respiratory dysfunction.
MV: Mechanical ventilation
P/F: PaO₂/FiO₂ ratio.
SA-AKI: Sepsis associated acute kidney injury.
RRT: Renal replacement therapy.
CRRT: Continuous renal replacement therapy

TABLE 12
Diagnostic test characteristics of the classification
and regression tree (CART) model predictive of sepsis
associated acute respiratory dysfunction on day 3 (D
3 SA-ARD) in the training, validation, and test datasets.
Diagnostic test characteristics in the training dataset (n = 375)

| | |
|---|---|
| Sensitivity | 0.91 (0.86, 0.94) |
| Specificity | 0.76 (0.68, 0.82) |
| Positive predictive value | 0.84 (0.78, 0.88) |
| Negative predictive value | 0.85 (0.78, 0.91) |
| Positive likelihood ratio | 3.73 (2.8, 4.93) |
| Negative likelihood ratio | 0.12 (0.08, 0.18) |

Diagnostic test characteristics in the validation dataset (n = 249)

| | |
|---|---|
| Sensitivity | 0.84 (0.77, 0.90) |
| Specificity | 0.73 (0.63, 0.82) |
| Positive predictive value | 0.84 (0.77, 0.89) |
| Negative predictive value | 0.74 (0.64, 0.82) |
| Positive likelihood ratio | 3.18 (2.26, 4.47) |
| Negative likelihood ratio | 0.21 (0.14, 0.31) |

Diagnostic test characteristics in the test dataset (n = 162)

| | |
|---|---|
| Sensitivity | 0.85 (0.80, 0.91) |
| Specificity | 0.64 (0.57, 0.72) |
| Positive predictive value | 0.64 (0.57, 0.72) |
| Negative predictive value | 0.85 (0.80, 0.91) |
| Positive likelihood ratio | 2.40 (1.79, 3.22) |
| Negative likelihood ratio | 0.23 (0.13, 0.41) |

Thus, the association between persistent sepsis-associated acute respiratory dysfunction and poor clinical outcomes in a large cohort of critically ill children with sepsis has been demonstrated herein. A clinico-endothelial biomarker model incorporating presence of day 1 SA-ARD, P/F ratio <250, sTM, and VCAM-1 concentrations was then derived and validated for prediction of patients who have persistent sepsis associated acute respiratory dysfunction. Finally, prognostic utility and reproducibility of this model was demonstrated in internal and external validation datasets.

While other groups have utilized statistical methods to develop risk models in adult septic patients at risk for acute hypoxemic respiratory failure, based on adult patients admitted to the intensive care unit with sepsis, no such work has been done on persistent sepsis associated acute respiratory dysfunction in a pediatric patient population. Regression analysis was used in those studies on adult populations to identify common lab and clinical variables independently associated with the development of acute hypoxemic respiratory failure. The final models had similar performance characteristics, with AUROCs values ranging from 0.79-0.81. The number of variables included in these models was between 6 and 13. The presently derived model achieved comparable AUROC values using only four variables in the present study on persistent sepsis associated acute respiratory dysfunction in a pediatric patient population.

The successful model derived as described herein comprised both biomarker and clinical variables. The model performance was poor overall without the clinical variables and lacked specificity without biomarkers. Subjects with D1 SA-ARD had an 80% chance of having D3 SA-ARD. Of those with D1 SA-ARD, subjects with a P/F ratio less than 250 had a very high rate of D3 SA-ARD (94.9%). The biomarkers added specificity to the model by identifying a subset of subjects with D1 SA-ARD but a P/F ratio greater than 250 that would go on to develop D3 SA-ARDs and were characterized by either high STM or low sTM and low VCAM 1. Subjects who do not have severe SA-ARD on day 1 but have persistence of SA-ARD on day 3 represent an ideal population to trial new early interventions that might modify the course of their disease.

Among the endothelial biomarkers evaluated, soluble thrombomodulin (sTM) was identified as having the highest odds of D3 SA-ARD among the endothelial biomarkers we tested. sTM is the product of the cleavage of thrombomodulin from the vascular endothelium, where it serves as a major component of the endogenous anti-coagulation system that prevents excessive coagulation. Given the role of endothelial dysfunction in the pathogenesis of sepsis and ARDS, sTM has been the subject of several studies showing that patients with elevated levels of sTM suffer worse outcomes. Y et, in randomized controlled trial of adults with sepsis and coagulopathy, patients who were treated with recombinant thrombomodulin (rTM) showed no benefit despite attempting enrichment by including only coagulopathic patients. Future pediatric trials can benefit from enrichment using our predictive model to define patients most likely to benefit from rTM.

An interesting finding of this study was that the univariate association between VCAM-1 concentrations and D3 SA-ARD was one of protection. VCAM-1 is a protein that is a member of the immunoglobulin superfamily and promotes vascular permeability and leukocyte migration to sites of inflammation and is thought to be a marker of endothelial activation. There is a paucity of literature describing VCAM-1 as a marker in pediatric sepsis or ARDS. VCAM-1 may be associated with an increasing number of organ dysfunctions in pediatric patients with sepsis and an independent association with indirect lung injury in pediatric sepsis. Results of both TreeNet® and CART analyses in this study showed that the lower odds of D3 SA-ARD with increments in VCAM-1 was largely restricted to patients with low sTM concentrations, although few patients in either cohort met these conditions. Nonetheless, the biological interaction between sTM and VCAM-1 on risk of sepsis associated ARDS is interesting.

This study has a number of strengths, including: 1) use of a large derivation cohort of pediatric septic shock patients and independent dataset to test model performance; (2) although TreeNet® method is informative by testing interactions between biomarkers on outcomes more systematically, the focus on CART methodology provides clear biomarker thresholds for risk-stratification and can be used to test model performance in other populations; (3) three distinct approaches were used to demonstrate robustness of the derived model, including cross-, hold-out-, and external validation.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the invention. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A method of classifying a patient with septic shock as high risk of sepsis-associated acute respiratory dysfunction (SA ARD) on day 3 (D3) or other than high risk of SA ARD on D3, the method comprising:
analyzing a dataset comprising biomarker expression levels of one or more endothelial biomarkers comprising soluble thrombomodulin (sTM) and vascular cell adhesion molecule 1 (VCAM-1), wherein the dataset is associated with a sample obtained from a pediatric patient with septic shock at a first time point, and wherein the dataset further comprises presence or absence of SA ARD on day 1 (D1) and PaO₂ to FIO₂ ratio for the patient;
determining whether the biomarker expression levels of each of the one or more endothelial biomarkers are greater than one or more pre-determined cut-off biomarker expression level, and determining whether the PaO₂ to FIO₂ ratio is less than a pre-determined cut-off PaO₂ to FIO₂ ratio; and
classifying the patient as high risk of SA ARD, or other than high risk of SA ARD, based on the presence or absence of SA ARD on D1, the determination of whether the expression levels of the one or more endothelial biomarkers are greater than the one or more pre-determined cut-off expression level, and/or the determination of whether the PaO₂ to FIO₂ ratio is less than a pre-determined cut-off PaO₂ to FIO₂ ratio.

2. The method of claim 1, wherein a classification of high risk of SA ARD comprises:
a) a presence of day 1 (D1) SA ARD, and a reduced PaO₂ to FIO₂ ratio;
b) a presence of day 1 (D1) SA ARD, a reduced PaO₂ to FIO₂ ratio, and an elevated level of sTM; or
c) a presence of day 1 (D1) SA ARD, a non-reduced PaO₂ to FIO₂ ratio, an elevated level of sTM, and a non-elevated level of VCAM-1;
and wherein a classification of other than high risk of SA ARD comprises:
d) an absence of D1 SA ARD; or
e) a presence of day 1 (D1) SA ARD, a non-reduced PaO₂ to FIO₂ ratio, a non-elevated level of sTM, and an elevated level of VCAM-1.

3. The method of claim 1, wherein biomarker expression levels comprise serum protein biomarker concentrations; or wherein biomarker expression levels are determined by quantifying serum protein biomarker concentrations and/or by cycle threshold (CT) values.

4. (canceled)

5. The method of claim 1, wherein the determined biomarker expression levels comprise expression levels of sTM and/or VCAM-1.

6. (canceled)

7. The method of claim 2, wherein biomarker levels are determined by serum protein biomarker concentration, and wherein:
a) an elevated level of sTM corresponds to a serum sTM concentration greater than about 15.37×10³ pg/ml; and
b) an elevated level of VCAM-1 corresponds to a serum VCAM-1 concentration greater than about 4.74×10⁶ pg/ml.

8. The method of claim 1, wherein the determination of whether the levels of the one or more biomarkers are non-elevated above a cut-off level comprises applying the biomarker expression level data to a decision tree comprising the two or more biomarkers.

9. (canceled)

10. The method of claim 1, wherein a classification other than high risk comprises a classification of low risk or intermediate risk; and/or wherein high risk of SA ARD by day 3 of septic shock or other than high risk of SA ARD on day 3 of septic shock is determined.

11. The method of claim 1, wherein SA ARD comprises direct lung injury and/or indirect lung injury, or wherein SA ARD comprises direct lung injury and/or indirect lung injury and further comprises cardiovascular, respiratory, renal, hepatic, hematologic, and/or neurologic dysfunction, and/or systemic inflammation and/or microvascular endothelial dysfunction, and/or low or no urine output, fluid overload with edema, increased need for supplemental oxygen or intubation and mechanical ventilation, need for dialysis, low blood pressure, multi-organ failure and/or death.

12. (canceled)

13. The method of claim 1, wherein the patient is undergoing continuous renal replacement therapy (CRRT).

14. (canceled)

15. The method of claim 1, wherein the classification is combined with one or more patient demographic data and/or clinical characteristics and/or results from other tests or indicia of septic shock and/or one or more additional biomarkers and/or platelet count and/or one or more additional population-based risk scores.

16. The method of claim 15, wherein the one or more additional biomarkers is selected from the group consisting of: interleukin-8 (IL-8), heat shock protein 70 kDa 1B (HSPA1B), and C-C Chemokine ligand 3 (CCL3); or wherein the one or more additional biomarkers is selected from the group consisting of: interleukin-8 (IL-8), heat shock protein 70 kDa 1B (HSPA1B), C-C Chemokine ligand 3 (CCL3), C-C Chemokine ligand 4 (CCL4), Granzyme B (GZMB), Interleukin-1 α (IL-1a), Matrix metallopeptidase 8 (MMP8), Angiopoietin-1 (Angpt-1), Angiopoietin-1 (Angpt-2), tyrosine kinase with immunoglobulin-like loops and epidermal growth factor homology domains 2 (Tie-2), Inter-Cellular Adhesion Molecule-1 (ICAM-1), P-selectin, E-selectin, and Platelet endothelial cell adhesion molecule-1 (PECAM-1); or wherein the patient demographic data and/or clinical characteristics and/or results from other tests or indicia of septic shock comprise at least one selected from the group consisting of: the septic shock causative organism, the presence or absence or chronic disease, and/or the age, gender, race, ethnicity, and/or co-morbidities of the patient.

17.-21. (canceled)

22. The method of claim 1, wherein the sample is obtained within the first hour of presentation with septic shock; or wherein the sample is obtained within the first 24 hours of presentation with septic shock.

23. (canceled)

24. The method of claim 1, further comprising administering a treatment comprising a therapy targeting endothelial dysfunction to a patient that is classified as high risk of SA ARD; or administering a treatment comprising a corticosteroid to a patient that is classified as other than high risk of SA ARD; or further comprising administering a treatment comprising one or more high risk therapy to a patient that is classified as high risk, or administering a treatment excluding a high risk therapy to a patient that is not high risk, to provide a method of treating a pediatric patient with septic shock.

25.-26. (canceled)

27. The method of claim 24, wherein the one or more high risk therapy comprises at least one selected from the group consisting of: biological and/or immune enhancing therapy, extracorporeal membrane oxygenation/life support, plasmapheresis, pulmonary artery catheterization, high volume continuous hemofiltration, non-corticosteroid therapy, adjuvant hemoperfusion, and/or plasma filtration and/or adsorption therapies.

28. (canceled)

29. The method of claim 1, wherein the patient is enrolled in a clinical trial; or wherein the patient is enrolled in a clinical trial and is classified as high risk.

30.-33. (canceled)

34. The method of claim 24, further comprising:
analyzing a second dataset associated with a second sample obtained from the treated patient at a second time point, wherein the dataset comprises biomarker expression levels of one or more endothelial biomarkers including sTM and VCAM-1 and further comprises presence or absence of SA ARD on day 1 (D1) and PaO₂ to FIO₂ ratio for the patient;
determining whether the biomarker expression levels of one or more biomarkers are greater than one or more pre-determined cut-off biomarker expression level, and determining whether the PaO₂ to FIO₂ ratio is less than a pre-determined cut-off PaO₂ to FIO₂ ratio;
classifying the patient as high risk of SA ARD, or other than high risk of SA ARD, based on the determination of whether the expression levels of each of the biomarkers are greater than the one or more pre-determined cut-off expression level, and/or the determination of whether the PaO₂ to FIO₂ ratio is less than a pre-determined cut-off PaO₂ to FIO₂ ratio; and
maintaining the treatment being administered if the patient's high risk classification has not changed, or changing the treatment being administered if the patient's high risk classification has changed.

35. The method of claim 34, wherein the second time point is at least 18 hours after the first time point; or wherein the second time point is in the range of 24 to 96 hours, or longer, after the first time point; or wherein the second time point is about 1 day, 2 days, 3 days, or longer, after the first time point; or wherein the first time point is at day 1, wherein day 1 is within 24 hours of a septic shock diagnosis, and the second time point is at day 3.

36.-42. (canceled)

43. The method of claim 34, wherein a patient not classified as high risk after the second time point is administered a treatment excluding a high risk therapy; or wherein the patient classified as high risk and administered one or more high risk therapy after the first time point is not classified as high risk after the second time point.

44. (canceled)

45. The method of claim 1, wherein one or more biomarker cut-off level is determined by one or more trained machine learning models based on a dataset generated from a cohort of pediatric patients with and without SA ARD; or wherein the data from the cohort of pediatric patients with and without SA ARD is provided to one or more machine learning models as input, and wherein the one or more trained machine learning model is based on a dataset generated from the biomarker cutoff levels in the patients of the cohort; or wherein one or more biomarker cut-off level is determined by a trained machine learning model, and wherein one or more machine learning models is used to classify the patient as high risk of SA ARD, or other than high risk of SA ARD.

46.-48. (canceled)

49. A diagnostic kit, test, or array comprising a reporter hybridization probe, and a capture hybridization probe specific for each of two or more mRNA, DNA, or protein biomarkers comprising STM and VCAM-1.

50. The diagnostic kit, test, or array of claim 49, wherein the biomarkers further comprise one or more of interleukin-8 (IL-8), heat shock protein 70 kDa 1B (HSPA1B), C-C Chemokine ligand 3 (CCL3), C-C Chemokine ligand 4 (CCL4), Granzyme B (GZMB), Interleukin-1 α (IL-1a), Matrix metallopeptidase 8 (MMP8), Angiopoietin-1 (Angpt-1), Angiopoietin-1 (Angpt-2), tyrosine kinase with immunoglobulin-like loops and epidermal growth factor homology domains 2 (Tie-2), Inter-Cellular Adhesion Molecule-1 (ICAM-1), P-selectin, E-selectin, and Platelet endothelial cell adhesion molecule-1 (PECAM-1); and/or further comprising a collection cartridge for immobilization of the hybridization probes; and/or wherein the reporter and the capture hybridization probes comprise signal and barcode elements, respectively.

51.-56. (canceled)
